SEQUENCE LISTING (26 sequences)

SEQ ID NO: 1 - protein, 744 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSEAAAKEAAAKEAAAKEAAAKEAAAKDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEFTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 744 aa, Artificial
  the sequence is synthesized
DIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEAAAKEAAAKEAAAKEAAAKEAAAKDIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSEFTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 717 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGSTSGSGKPGSGEGSTKGQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSEFTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 717 aa, Artificial
  the sequence is synthesized
DIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSDIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGSTSGSGKPGSGEGSTKGQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEFTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2232 bp, Artificial
  the sequence is synthesized
gatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggtggtggtggttctggcggcggcggctccggtggtggtggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcgaagccgctgctaaggaagccgctgctaaggaagccgctgctaaggaagccgctgctaaggaagccgctgctaaggacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggtggtggtggttctggcggcggcggctccggtggtggtggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - DNA, 2232 bp, Artificial
  the sequence is synthesized
gacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggtggtggtggttctggcggcggcggctccggtggtggtggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaagccgctgctaaggaagccgctgctaaggaagccgctgctaaggaagccgctgctaaggaagccgctgctaaggatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggtggtggtggttctggcggcggcggctccggtggtggtggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcgaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 7 - DNA, 2151 bp, Artificial
  the sequence is synthesized
gatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggcggcggaggttctgacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggcagcacaagcggctctggcaagcctggatctggcgagggctctaccaagggccaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcaggcggcggaggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcgaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - DNA, 2151 bp, Artificial
  the sequence is synthesized
gacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggcggcggaggttctgatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggcagcacaagcggctctggcaagcctggatctggcgagggctctaccaagggccaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcggcggcggaggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 9 - protein, 520 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSEAAAKEAAAKEAAAKEAAAKEAAAKDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSS

SEQ ID NO: 10 - protein, 520 aa, Artificial
  the sequence is synthesized
DIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEAAAKEAAAKEAAAKEAAAKEAAAKDIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSS

SEQ ID NO: 11 - protein, 493 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGSTSGSGKPGSGEGSTKGQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSS

SEQ ID NO: 12 - protein, 493 aa, Artificial
  the sequence is synthesized
DIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSDIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGSTSGSGKPGSGEGSTKGQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSS

SEQ ID NO: 13 - protein, 21 aa, Homo sapiens
  the sequence is synthesized
MALPVTALLLPLALLLHAARP

SEQ ID NO: 14 - protein, 222 aa, Artificial
  the sequence is synthesized
TTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 993 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSFETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAAAEGRGSLLTCGDVEENPGPSGATMALPVTALLLPLALLLHAARPGSDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEFTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 992 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSFETTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAAAEGRGSLLTCGDVEENPGPSGATMALPVTALLLPLALLLHAARPGSDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEFTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 992 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSFETTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAAAEGRGSLLTCGDVEENPGPSGATMALPVTALLLPLALLLHAARPGSDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEFTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 991 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSSFETTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAAAEGRGSLLTCGDVEENPGPSGATMALPVTALLLPLALLLHAARPGSDIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSSEFTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 2982 bp, Artificial
  the sequence is synthesized
gatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggtggtggtggttctggcggcggcggctccggtggtggtggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcttcgaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctgagggcagaggaagtcttctaacatgcggtgacgtggaggagaatcccggcccttccggagccaccatggccttaccagtgaccgccttgctcctgccgctggccttgctgctccacgccgccaggccgggatccgacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggtggtggtggttctggcggcggcggctccggtggtggtggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 20 - DNA, 2976 bp, Artificial
  the sequence is synthesized
gatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggtggtggtggttctggcggcggcggctccggtggtggtggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcttcgaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctgagggcagaggaagtcttctaacatgcggtgacgtggaggagaatcccggcccttccggagccaccatggccttaccagtgaccgccttgctcctgccgctggccttgctgctccacgccgccaggccgggatccgacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggtggtggtggttctggcggcggcggctccggtggtggtggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 2976 bp, Artificial
  the sequence is synthesized
gatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggtggtggtggttctggcggcggcggctccggtggtggtggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcttcgaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctgagggcagaggaagtcttctaacatgcggtgacgtggaggagaatcccggcccttccggagccaccatggccttaccagtgaccgccttgctcctgccgctggccttgctgctccacgccgccaggccgggatccgacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggtggtggtggttctggcggcggcggctccggtggtggtggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - DNA, 2973 bp, Artificial
  the sequence is synthesized
gatattgtgctgacccagagccccaagttcatgagcaccagcgtgggcgatagagtgagcgtgacctgcaaggcaagccagaacgtgggaacaaacgtggcctggtaccaacagaaacccggccaaagccctaagcccctgatttacagcgccacctacagaaatagcggcgtgcccgacagatttacaggaagcggcagcggaaccgatttcacactgaccatcaccaacgtgcagagcaaagacctggccgactacttctgccagcagtacaacagatacccctacaccagcggaggaggaacaaagctggagatcaagagaggtggtggtggttctggcggcggcggctccggtggtggtggttctcaagtgcaactgcaacagagcggagccgaactggtgagacccggaagcagcgtgaagatcagctgcaaggcttccggctacgcctttagcagctactggatgaactgggtgaagcagagacctggacagggactggaatggatcggccagatttaccctggagacggcgacacaaactacaacggcaagttcaagggccaagctacactgaccgccgacaaaagcagcagcaccgcctatatgcagctgagcggactgaccagcgaagatagcgctgtgtacttctgcgccagaaagaccatcagcagcgtggtggacttctacttcgactactggggacaaggcaccaccctgacagtgagcagcttcgaaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctgagggcagaggaagtcttctaacatgcggtgacgtggaggagaatcccggcccttccggagccaccatggccttaccagtgaccgccttgctcctgccgctggccttgctgctccacgccgccaggccgggatccgacattgagctcacccagtctccatcctccctagctgtgtcagttggagagaaggttactatgagctgcaagtccagtcagagccttttatatagtagcaatcaaaagaactatttggcctggtaccagcagaaaccagggcagtctcctaaactgctgatttactgggcatccactagggaatctggggtccctgatcgcttcacaggcagtggatctgggacagatttcactctcaccgtcagcagtgtgaaggctgaagacctggcagtttattactgtcagcaatcttatagttatccattcacattcggctcgggcaccaagctggaaatcaaacggggtggtggtggttctggcggcggcggctccggtggtggtggttctcaggtcaaactgcagcagtcaggacctgaactggtgaagcctggggcctcagtgaagatttcctgcaaagcttctggctacgatttcagtatttcttggatgaactgggtgaggcagaggcctggacagggtcttgagtggattggacggatttatcctggagatggagatagtaactacaatgggaagttcgagggcaaggccacactgactgcagacaaatcctccagcacagcctacatgcagctcagcggcctgacctctgtggactctgcggtctatttttgtgcaagaaccaccactatgattgccctctatgctatggactactggggccaagggaccacggtcaccgtctcctcagaattcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 23 - protein, 245 aa, Artificial
  the sequence is synthesized
DIVLTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSQVQLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTLTVSS

SEQ ID NO: 24 - protein, 250 aa, Artificial
  the sequence is synthesized
DIELTQSPSSLAVSVGEKVTMSCKSSQSLLYSSNQKNYLAWYQQKPGQSPKLLIYWASTRESGVPDRFTGSGSGTDFTLTVSSVKAEDLAVYYCQQSYSYPFTFGSGTKLEIKRGGGGSGGGGSGGGGSQVKLQQSGPELVKPGASVKISCKASGYDFSISWMNWVRQRPGQGLEWIGRIYPGDGDSNYNGKFEGKATLTADKSSSTAYMQLSGLTSVDSAVYFCARTTTMIALYAMDYWGQGTTVTVSS

SEQ ID NO: 25 - protein, 223 aa, Artificial
  the sequence is synthesized
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 18 aa, Artificial
  the sequence is synthesized
EGRGSLLTCGDVEENPGP

(19) United States
(12) Patent Application Publication
(10) Pub. No.: US 2023/0272069 A1
(43) Pub. Date: Aug. 31, 2023

(54) Chimeric Antigen Receptor Targeting CD22 and CD19 and Application thereof

(71) Applicant: Institute of Hematology and Blood Diseases Hospital, Chinese Academy of Medical Sciences, Tianjin (CN)

(72) Inventors: Jianxiang WANG, Tianjin (CN); Min WANG, Tianjin (CN); Yu ZHANG, Tianjin (CN); Yingxi XU, Tianjin (CN); Qing RAO, Tianjin (CN); Xiaolong LIAO, Tianjin (CN)

(21) Appl. No.: 17/681,855

(22) Filed: Feb. 28, 2022

(51) Int. Cl. C07K 16/28; C07K 14/725; C07K 14/705; C07K 14/535; A61P 35/02

(52) U.S. Cl. CPC C07K 16/2803; A61P 35/02; C07K 14/535; C07K 14/7051; C07K 14/70517; C07K 14/70521; C07K 14/70578; A61K 38/00; C07K 2317/622; C07K 2319/02; C07K 2319/03

(57) ABSTRACT

The present invention discloses a nucleic acid molecule for encoding a chimeric antigen receptor targeting CD22 and CD19. The chimeric antigen receptor of the present invention can be used for treatment of CD19⁺ and CD22⁺ B-cell hematological tumors, as well as combined treatment with CD19 CAR-T cells or CD22 CAR-T cells.

### BACKGROUND OF THE INVENTION

The present invention relates to the technical field of biomedicine, in particular to a chimeric antigen receptor targeting CD22 and CD19 and application thereof.

As an immunotherapy strategy, chimeric antigen receptor (CAR)-modified T cells have received extensive attention and application in tumor treatment. The structure of a CAR is generally composed of four parts: an extracellular targeting linking region (usually being a single-chain antibody with an antigen recognition function), a hinge region, a transmembrane region and an intracellular signal transduction region. At present, according to the number of costimulatory molecules added to the intracellular signal transduction region, CARs are divided into first generation (without costimulatory molecules), second generation (with one costimulatory molecule) and third generation (with two costimulatory molecules). The second-generation CARs are currently the most widely used.

B-cell malignancies such as B-cell lymphocytic leukemia (B-ALL) and lymphoma are malignant diseases caused by abnormal clonal proliferation of B-lymphocytes. Despite the remarkable effect of current chemotherapy, 15% of childhood B-ALL patients and 60% of adult B-ALL patients have a poor prognosis due to chemoresistance; and 15% of lymphoma patients still relapse after first-line therapy, and 50% of these patients eventually relapse after hematopoietic stem cell transplantation. Therefore, the search for effective cellular immunotherapy to treat B-cell malignancies has always been a research hotspot in the field of hematology.

CD19 and CD22 are antigenic molecules unique to the surface of B-lymphocytes. CD19 is expressed on the surface of almost all B-ALL and lymphoma cells, on the surface of normal hematopoietic cells of the B lineage, but not in normal non-hematopoietic tissues. CD22 is a B-lineage differentiation antigen, which is expressed in various stages of B cell development. After B cells differentiate into plasma cells, CD22 is no longer expressed. 60% to 80% of B cell malignant tumors express CD22, and 90% or more of diffuse large B-cell lymphomas (DLBCLs) and follicular lymphomas (FLs) are CD22 positive. Almost all B-precursor cell acute lymphoblastic leukemias (B-ALL) express CD22. Chronic lymphocytic leukemia (CLL) and hairy cell leukemia (HCL) also have high levels of CD22 expression. Similar to a CD19 antigen, CD22 is also restricted to B cells, not expressed in other parenchymal cells, nor in hematopoietic stem cells. Therefore, as a B-cell tumor antigen with high specificity, CD22 has become the main therapeutic target in B-cell malignant tumors.

At present, although CD19-CAR-T has achieved good efficacy in the treatment of refractory and relapsed B-ALL, there are still patients with CD19 antigen mutation or loss and other abnormalities during treatment, resulting in the failure of CD19-CAR-T cells to recognize and kill B-ALL cells, leading to disease recurrence. CD22 and CD19 have extensive co-expression on the surface of tumor cells, and CD22 remains after the loss of a CD19 antigen. Therefore, CAR-T cells targeting CD19 and CD22 can effectively avoid antigenic variation, reduce relapse, and improve the efficacy of anti-B-lineage malignant tumors.

However, it has been found that if antigen-binding sites targeting CD19 and CD22 are inserted into a vector by a simple incorporation, the killing effect after expression is not good. Therefore, it is necessary to find a specific incorporating mode of the antigen-binding sites of CD19 and CD22 to improve the effect of killing tumor cells.

### BRIEF SUMMARY OF THE INVENTION

In one aspect of the present invention, a dual-target CAR targeting CD22 and CD19 simultaneously and application thereof are provided for relapse or ineffectiveness during CD19 CAR-T treatment caused by an antigen escape mechanism of B-ALL in the prior art.

The technical solution provided by the present invention is:

A nucleic acid molecule for encoding a CAR targeting CD22 and CD19, wherein the CAR includes an extracellular region, a transmembrane region and an intracellular signal transduction region, and the extracellular region encoded by the nucleic acid molecule includes CD22 and CD19 binding domains consisting of a CD22 single-chain fragment variable (CD22 scFv) and a CD19 single-chain fragment variable (CD19 scFv);

and the CD22 scFv and the CD19 scFv are arranged according to an amino acid sequence shown in SEQ ID No.9, an amino acid sequence shown in SEQ ID No.10, an amino acid sequence shown in SEQ ID No.11, or an amino acid sequence shown in SEQ ID No. 12 in order.

In the present invention, CD22 positive (CD22⁺) tumor cells and CD19 positive (CD19⁺) tumor cells are exposed to a CAR of 19-22 shown in SEQ ID No.1, a CAR of 22-19 shown in SEQ ID No.2, a CAR of 19×22 shown in SEQ ID No.3, a CAR of 22×19 shown in SEQ ID No.4, a CAR of 1922-BB shown in SEQ ID No.15, a CAR of 1922-28B shown in SEQ ID No.16, a CAR of 1922-B28 shown in SEQ ID No.17, or a CAR of 1922-2828shown in SEQ ID No.18 modified T cells (CAR-T) to activate corresponding CAR-T cells and produce cytotoxic effects. However, cells expressing neither CD22 nor CD19 cannot activate CAR-T cells to respond. Therefore, CAR-T cells prepared with CD19scFv-CD22scFv (shown in SEQ ID No. 1), CD22scFv-CD19 scFv (shown in SEQ ID No. 2), CD19 V_{L}-CD22 V_{L}-CD22 V_{H}-CD19 V_{H} (shown in SEQ ID No. 3), CD22 V_{L}-CD19 V_{L}-CD19V_{H}-CD22V_{H} (shown in SEQ ID No. 4), CD19scFv (shown in SEQ ID No. 23) and CD22scFv (shown in SEQ ID No. 24) as antigen recognition regions do not produce off-target effects on cells that do not express CD19 or CD22 while recognizing and killing CD22⁺ tumor cells and CD19⁺ tumor cells.

In the present invention, the inventors also found that the sequences order and rearrangement of CD22 scFv and CD19 scFv have a great influence on the effect of killing tumor cells after their incorporation. For example, in one embodiment of the present invention, the inventors verified that a linking mode of 22-19 CAR-T has a more significant killing effect.

The sequences of CD22 scFv and CD19 scFv of the present invention can be prepared according to a conventional method according to the description in SEQ ID No. 1-4,15-19, for example, methods described in the patent with the patent number of ZL201510233748.0 and the patent with the application number of 201810549259.X.

In the present invention, the amino acid sequences of CD19scFv-CD22scFv, CD22scFv-CD19 scFv, CD19 V_{L}-CD22 V_{L}-CD22 V_{H}-CD19 V_{H}, CD22 V_{L}-CD19 V_{L}-CD19V_{H}-CD22V_{H}, CD19 scFv and CD22 scFv can be subjected to random or engineered point mutations in a suitable manner. The purpose can be, for example, obtaining better affinity and/or dissociation properties. The mutated amino acid sequences are all included in the protection scope of the present invention.

In the present invention, light and heavy chains of the same or different antibodies of CD22 scFv and CD19 scFv are incorporated by linking peptides (linkers), and any linking peptides of a suitable length or property or combination of linking peptides can achieve the purpose of the present invention.

In the present invention, the nucleic acid molecule can encode a signal peptide. The signal peptide can guide the transfer of an antigen recognition region and a hinge region to the outside of a cell. Any suitable signal peptide or combination of signal peptides can achieve the purpose of the present invention.

Preferably, in one embodiment of the present invention, the extracellular region encoded by the nucleic acid molecule of the present invention further includes a signal peptide constructed at an amino terminal of the CAR or an amino acid sequence having 90%-99% homology with the signal peptide. The signal peptide is a signal peptide sequence in CD8α or GM-CSF.

More preferably, the signal peptide is a signal peptide shown in SEQ ID NO.13.

In one embodiment of the present invention, the CD22 and CD19 binding domains encoded by the nucleic acid molecule are connected to the transmembrane region encoded by the nucleic acid molecule through a hinge region. Any suitable hinge region sequence can achieve the purpose of the present invention. Preferably, in one embodiment of the present invention, the hinge region is CD8α.

In the present invention, the nucleic acid molecule further encodes a transmembrane domain. Any suitable transmembrane domain can achieve the purpose of the present invention. Preferably, in one embodiment of the present invention, the transmembrane region is a transmembrane domain selected from the following proteins or an amino acid sequence having 90%-99% homology with the proteins: α, β or ζ chains of T cell receptors, CD3ε, CD45, CD4, CD5, CD8, CD9, CD16, CD22, CD33, CD37, CD64, CD80, CD86, CD134, CD137 or CD154.

In the present invention, the intracellular signal transduction region encoded by the nucleic acid molecule further includes a costimulatory factor.

Preferably, the costimulatory factor is one or more of functional signal domains obtained by selecting from the following proteins or the amino acid sequence having 90%-99% homology with the proteins: MHC class I molecules, TNF receptor proteins, immunoglobulin-like proteins, cytokine receptors, integrins, lymphocyte activation signaling molecules, activated NK cell receptors, BTLA, Tollligand receptors, OX40, CD2, CD7, CD27, CD28, CD30, CD40, CDS, ICAM-1, LFA-1, 4-1BB, B7-H3, CD278, GITR, BAFFR, LIGHT, HVEM, KIRDS2, SLAMF7, NKp80, NKp44, NKp30, NKp46, CD19, CD4, CD8α, CD8β, IL2Rβ, IL2Rγ, IL7Rα, ITGA4, VLA1, CD49α, IA4, CD49D, ITGA6, VLA6, CD49f, ITGAD, CD11d, ITGAE, CD103, ITGAL, CD11α, ITGAM, CD11b, ITGAX, CD11c, CD29, ITGB1, ITGB2, CD18, ITGB7, NKG2D, NKG2C, TNFR2, CD226, CD84, CD96, CEACAM1, CRTAM, CD229, CD160, PSGL1, CD100, CD69, SLAMF6, SLAM, BLAME, CD162, LTBR, LAT, GADS or SLP-76.

More preferably, the costimulatory factor is CD28 or 4-1BB or an amino acid sequence having 90%-99% homology therewith.

At the same time, the nucleic acid molecule of the present invention further encodes any suitable intracellular signal domain. It can be a CD3ζ intracellular signaling structure and an amino acid sequence having 90%-99% homology therewith.

Preferably, the CAR encoded by the nucleic acid molecule of the present invention takes a structure formed by incorporating CD19scFv-CD22scFv, CD22scFv-CD19 scFv, CD19 V_{L}-CD22 V_{L}-CD22 V_{H}-CD19 V_{H}, CD22 V_{L}-CD19 V_{L}-CD19V_{H}-CD22V_{H},CD19 scFv and CD22 scFv antigen recognition regions, a CD8α hinge region, a transmembrane region, and a 4-1BB or CD28 or CD3ζ intracellular signal domain as a signal transduction domain, and its sequence is shown in SEQ ID NO.5-8 and SEQ ID NO.19-22.

In addition, any peptide chain can be inserted as a spacer at a suitable position between the above antigen recognition region, hinge region, transmembrane region and intracellular signal region, and the peptide chain can be an oligopeptide or a polypeptide.

The above nucleic acid molecule can be prepared by known techniques such as chemical synthesis or PCR amplification based on base sequences of the above domains such as the antigen recognition region, the hinge region, the transmembrane region and the intracellular signal region. Typically, codons encoding amino acids of the above domains can be optimized to optimize their expression in host cells. Information of the above base sequences can be obtained by searching published literature or databases such as NCBI (https://www.ncbi.nlm.nih.gov/).

In one embodiment of the present invention, the mouse anti-human CD22 monoclonal antibody hybridoma cell line (HIB22) and the mouse anti-human CD19 monoclonal antibody hybridoma cell line (HIB19) used by the inventors are developed by the applicant- Institute of Hematology & Blood Diseases Hospital, Chinese Academy of Medical Sciences (CAMS), and the monoclonal antibodies produced by the hybridomas are commercially available for the detection and diagnosis of CD22⁺ and CD19⁺ acute lymphoblastic leukemia (ALL) and lymphoma, respectively.

In another aspect of the present invention, a CAR is provided. The CAR is encoded by the above nucleic acid molecule.

An extracellular region of the above CAR includes CD22 and CD19 binding domains consisting of a CD22 scFv and a CD19 scFv.

and the CD22 scFv and the CD19 scFv are arranged according to an amino acid sequence shown in SEQ ID No.9, an amino acid sequence shown in SEQ ID No.10, an amino acid sequence shown in SEQ ID No.11, or an amino acid sequence shown in SEQ ID No. 12 in order.

Preferably, the CAR of the present invention takes a structure formed by incorporating CD19scFv-CD22scFv, CD22scFv-CD19 scFv, CD19 V_{L}-CD22 V_{L}-CD22 V_{H}-CD19 V_{H}, CD22 V_{L}-CD19 V_{L}-CD19V_{H}-CD22V_{H}, CD19 scFv and CD22 scFv antigen recognition regions, a CD8α hinge region, a transmembrane region, and a 4-1BB or CD28 or CD3ζ intracellular signal domain as a signal transduction domain, and an amino acid sequence is shown in a sequence table of SEQ ID NO.1-4 and SEQ ID NO.15-18.

In another aspect of the present invention, a vector is provided, including the above nucleic acid molecule.

In the present invention, the above vector may be a linear vector or a circular vector. The vector may be a non-viral vector such as a plasmid, a viral vector, or a vector using a transposon. The vector may contain regulatory sequences such as promoters and terminators, as well as marker sequences such as drug resistance genes and reporter genes. In addition, the above vector may also contain a sequence encoding a suicide gene, and the number of CAR-T cells in the body may be controlled by administering a substance that activates the suicide gene according to the treatment process.

Examples of the viral vector include retroviral vectors, lentiviral vectors, adenoviral vectors, adeno-associated viral vectors, and so on. In one embodiment of the present invention, a lentiviral expression vector is used.

In another aspect of the present invention, a cell is provided, including the above nucleic acid molecule, the above CAR or the above vector.

In one embodiment of the present invention, the cell is human T cells. The T cells may be from body fluids such as blood and bone marrow, or from tissues such as spleen, thymus, and lymph, or from cancer tissues such as primary tumors, metastatic tumors, and cancerous ascites, and are obtained after separation and purification. Meanwhile, the T cells may be CD4⁺ T cells, CD8⁺ T cells, αβ T cells or γδ T cells. The T cells may be replaced by NK cells in a suitable manner, which are also considered to be included in the protection scope of the present invention.

On another aspect, the present invention provides application of the above nucleic acid molecule in preparation of B-cell hematological tumor drugs.

On another aspect, the present invention provides application of the above CAR in preparation of B-cell hematological tumor drugs.

On another aspect, the present invention provides application of the above vector in preparation of B-cell hematological tumor drugs.

On another aspect, the present invention provides application of the above cell in preparation of B-cell hematological tumor drugs.

Preferably, the above application is application in the preparation of the anti-B cell hematological tumor drugs for simultaneous expression of CD19 and CD22 or for recurrence or ineffectiveness of a patient after CD19 CAR-T treatment due to antigenic variation.

The above ineffectiveness means that after CD19 CAR-T treatment, the patient has no response to CD19 CAR-T due to CD19 antigen loss, mutation, etc., and the treatment is ineffective.

As long as they express CD19 and CD22 in the pathological process, the above B-cell hematological tumors include, but are not limited to, B-cell lymphoma, ALL, and so on. Preferably, the B-cell hematological tumors are B-cell lymphoma or B-ALL.

In another aspect of the present invention, a pharmaceutical composition is provided, including the above nucleic acid molecule, the above CAR, the above vector or the above cell, and a pharmaceutically acceptable vector.

In addition to the above components, the pharmaceutical composition of the present invention may also contain any pharmaceutically acceptable additives, such as physiological saline, a cell culture medium, glucose, water for injection, glycerol, ethanol and their combinations, stabilizers, surfactants, preservatives, isotonic agents, etc.

Similarly, the pharmaceutical composition of the present invention may also be used in combination with other suitable anticancer agents, for example, vincristine, daunorubicin, asparaginase, cyclophosphamide, prednisone, and so on.

Preferably, the pharmaceutical composition of the present invention further includes a nucleic acid molecule for encoding a CD19 and CD22 CAR, the CD19 and CD22 CAR, a vector containing the nucleic acid molecule for encoding the CD19 and CD22 CAR or a cell containing the CD19 and CD22 CAR.

The nucleic acid molecule for encoding the CD19 and CD22 CAR, the CD19 and CD22 CAR, the vector containing the nucleic acid molecule for encoding the CD19 and CD22 CAR or the cell containing the CD19 and CD22 CAR may be any suitable nucleic acid molecule for encoding the CD19 and CD22 CAR, CD19 and CD22 CAR, vector containing the nucleic acid molecule for encoding the CD19 and CD22 CAR or cell containing the CD19 and CD22 CAR.

In another aspect of the present invention, application of the above nucleic acid molecule in treatment of B-cell hematological tumors is provided.

In another aspect of the present invention, application of the above CAR in treatment of B-cell hematological tumors is provided.

In another aspect of the present invention, application of the above vector in treatment of B-cell hematological tumors is provided.

In another aspect of the present invention, application of the above cell in treatment of B-cell hematological tumors is provided.

In another aspect of the present invention, application of the above pharmaceutical composition in treatment of B-cell hematological tumors is provided.

The present invention has the beneficial effects:

In the present invention, the light and heavy chains of mouse anti-human CD22 scFv and mouse anti-human CD19 scFv are synthesized and rearranged by nucleic acid molecule chemical synthesis or PCR technology to obtain CD19 scFv-CD22 scFv, CD22 scFv-CD19 scFv, CD19 V_{L}-CD22 V_{L}-CD22 V_{H}-CD19 V_{H} and CD22 V_{L}-CD19 V_{L}-CD19V_{H}-CD22V_{H}, cloned into the lentiviral expression vector containing the signal peptide and CD8α-4-1 BB/CD28-CD3ζ, and packaged into lentiviral vectors carrying encoding genes CD19scFv-CD22scFv-CD8α-4-1 BB-CD3ζ, CD22scFv-CD19scFv-CD8α-4-1BB-CD3ζ, CD19 V_{L}-CD22 V_{L}-CD22 V_{H}-CD19 V_{H}-CD8α-4-1BB-CD3ζ, CD22 V_{L}-CD19 V_{L}-CD19 V_{H}-CD22 V_{H}-CD8α-4-1BB-CD3ζ, CD19 scFv-CD8α-4-1BB-CD3ζ-T2A-CD22 scFv-CD8α-4-1BB-CD3ζ, CD19 scFv-CD8α-CD28-CD3ζ-T2A-CD22 scFv-CD8α-4-1BB-CD3ζ, CD19 scFv-CD8α-4-1BB-CD3ζ-T2A-CD22 scFv-CD8α-CD28-CD3ζ and CD19 scFv-CD8α-28-CD3ζ-T2A-CD22 scFv-CD8α-28-CD3ζ. The T cells are infected with lentivirus to express the above CAR. Through flow cytometry, degranulation assays, and ELISA for cytokines secreted by T cells, it was proved that the CAR modified T cells have a strong killing effect on CD19⁺, CD22⁺, and CD19⁺/CD22⁺ B-cell lymphoma cells and B-cell lymphocytic leukemia cells, and have almost no killing effect on cells that do not express CD19 and CD22, effectively preventing off-target effects. The CAR of the present invention can be used for the treatment of CD19⁺ and CD22⁺ B-cell hematological tumors, as well as the combined treatment with CD19 CAR-T cells or CD22 CAR-T cells.

### BRIEF DESCRIPTION OF THE DRAWINGS

FIG. 1A and FIG. 1B are restriction endonuclease digestion segment electrophoresis identification diagrams of lentiviral expression vectors of CD19scFv-CD22scFv-CD8α-4-1 BB-CD3ζ, CD22scFv-CD19scFv-CD8α-4-1 BB-CD3ζ, CD19 V_{L}-CD22 V_{L}-CD22 V_{H}-CD19 V_{H}-CD8α- 4-1 BB-CD3ζ and CD22 V_{L}-CD19 V_{L}-CD19 V_{H}-CD22 V_{H}-CD8α-4-1 BB-CD3ζ in an embodiment of the present invention; in FIG. 1A, 1 is a 15kb nucleic acid molecular weight marker lane, 2 is a DNA segment (2314bp) and a vector segment (7228bp) encoding CD19 scFv-CD22 scfv-CD8α-4-1BB-CD34 obtained by double digestion of a lentiviral expression plasmid CD19 scFv-CD22 scFv-CD8α-4-1 BB-CD3ζ with endonucleases Nhel and Notl, and 3 is a DNA segment (2314bp) and a vector segment (7228bp) encoding CD22 scFv-CD19 scfv-CD8α-4-1 BB-CD3ζ obtained by double digestion of a lentiviral expression plasmid CD22 scFv-CD19 scFv-CD8α-4-1BB-CD3ζ with endonucleases Nhe I and Not I; and in FIG. 1B, 4 is a 15kb nucleic acid molecular weight marker lane, 5 is a DNA segment (2233bp) and a vector segment (7234bp) encoding CD19V_{L}-CD22V_{L}-CD22V_{H}-CD19V_{H}-CD8α-4-1BB-CD3ζ obtained by double digestion of a lentiviral expression plasmid CD19 V_{L}-CD22V_{L}-CD22V_{H}-CD19V_{H}-CD8α-4-1BB-CD3ζ with endonucleases Nhel and Notl, and 6 is a DNA segment (2233bp) and a vector segment (7234bp) encoding CD22 V_{L}-CD19 V_{L}-CD19V_{H}-CD22V_{H}-CD8α-4-1BB-CD3ζ obtained by double digestion of a lentiviral expression plasmid CD22 V_{L}-CD19 V_{L}-CD19V_{H}-CD22V_{H}-CD8α-4-1 BB-CD3ζ with endonucleases Nhel and Notl.

FIG. 2 is a schematic diagram with a lentiviral expression vector of CD22 scFv-CD19 scFv-CD8α-4-1 BB-CD3ζ as an example in an embodiment of the present invention, where a counterclockwise sequence is a forward gene segment, and a clockwise sequence is a reverse gene segment.

FIG. 3A shows results(represented as 19-22 CAR-T, 22-19 CAR-T, 19×22 CAR-T, 22×19 CAR-T, respectively) of testing expression of CAR molecules in dual CAR-target CD19 scFv-CD22 scFv-CD8α-4-1BB-CD3ζ, CD22 scFv-CD19 scFv-CD8α-4-1BB-CD3ζ, CD19 V_{L}-CD22 V_{L}-CD22 V_{H}-CD19 V_{H}-CD8α-4- 1BB-CD3ζ and CD22 V_{L}-CD19 V_{L}-CD19 V_{H}-CD22 V_{H}-CD8α-4-1BB-CD3ζ modified T cells constructed in an embodiment of the present invention using flow cytometry, and also shows results (represented as 19 CAR-T and 22 CAR-T, respectively) of expression of CAR molecules in single CAR-target CD19 scFv-CD8α-4-1 BB-CD3ζ and CD22 scFv-CD8α-4-1 BB-CD3ζ modified T cells, to compare functions of single-target and dual-target CAR-T. GFP is expression of marker proteins carried by a vector. F(ab′)₂ is rabbit anti-mouse IgG to detect (or label) CD22 scFv and CD19 scFv on the surface of T cells. FIG. 3B shows results (represented as1922-BB, 1922-28B, 1922-B28, and 1922-2828, respectively) of testing expression of CAR molecules in dual-target CD19 scFv-CD8α-4-1BB-CD3ζ-T2A-CD22 scFv-CD8α-4-1 BB-CD3ζ, CD19 scFv-CD8α-CD28-CD3ζ-T2A-CD22 scFv-CD8α-4-1 BB-CD3ζ, CD19 scFv-CD8α-4-1BB-CD3ζ-T2A-CD22 scFv-CD8α-CD28-CD3ζ and CD19 scFv-CD8α-28-CD3ζ-T2A-CD22 scFv-CD8α-28-CD3ζ-modified T cells constructed in an embodiment of the present invention using flow cytometry.

FIG. 4A and FIG. 4B show results of testing expression of target cells applied in an embodiment of the present invention: a Burkitt lymphoma cell line Namalwa, a chronic granulocytic leukemia cell line MV4-11-CD19 infected with CD19 antigen molecules, a chronic granulocytic leukemia cell line MV4-11-CD22 infected with CD22 antigen molecules, and CD19 and CD22 antigen molecules in wild-type MV4-11 cells by using flow cytometry, where FIG. 4A is expression rate of CD19 antigen molecules; and FIG. 4B is expression rate of CD22 antigen molecules.

FIG. 5A to FIG. 5L show results of residual tumor cells after co-culture of T cells with target tumor cells in an embodiment of the present invention using flow cytometry, where vector-T is a control group of transfected empty vector-T cells; 19CAR-T is an experimental group of CD19 scFv-CD8α-4-1BB-CD3ζ-modified T cells, 22CAR-T is an experimental group of CD22 scFv-CD8α-4-1 BB-CD3ζ-modified T cells, 19-22CAR-T is an experimental group of CD19 scFv-CD22 scFv-CD8α-4-1BB-CD3ζ-modifiedT cells, 22-19 CAR-T is an experimental group of CD22 scFv-CD19 scFv-CD8α-4-1BB-CD3ζ-modifiedT cells, 19×22 CAR-T is an experimental group of CD19 V_{L}-CD22V_{L}-CD22V_{H}CD19V_{H}CD8a-4-1BB-CD3ζ-modified T cells, and 22×19 CAR-T is an experimental group of CD22 V_{L}-CD19V_{L}-CD19V_{H}CD22V_{H} CD8α-4-1 BB-CD3ζ-modified T cells. FIG. 5A and FIG. 5B respectively show percentages of residual tumor cells after co-culture of vector-T, 19 CAR-T, 22 CAR-T, 19-22 CAR-T and 22-19 CAR-T with target tumor cells for 24 hours at different effector: target ratios (E:T ratio) of 1:8, 1:4, 1:2 and 1:1, respectively, and a schematic flow diagram of residual tumor cells percentages after co-culture for 48 hours at E:T ratio of 1:1 when the target cells were Namalwa cell lines. FIG. 5C and FIG. 5D respectively show percentages of residual tumor cells after co-culture of vector-T, 19 CAR-T, 22 CAR-T, 19-22 CAR-T and 22-19 CAR-T with target tumor cells for 24 hours at E:T ratio of 1:8, 1:4, 1:2 and 1:1, respectively, and a schematic flow diagram of percentages of residual tumor cells after co-culture for 48 hours at E:T ratio of 1:1 when the target cells were MV4-11-CD19 cell lines. FIG. 5E and FIG. 5F respectively show percentages of residual tumor cells after co-culture of vector-T, 19 CAR-T, 22 CAR-T, 19-22 CAR-T and 22-19 CAR-T with target cells for 24 hours at E:T ratio of 1:8, 1:4, 1:2 and 1:1, respectively, and a schematic flow diagram of percentages of residual tumor cells after co-culture for 48 hours at E:T ratio of 1:1 when the target cells were MV4-11-CD22 cell lines. FIG. 5G and FIG. 5H respectively show percentages of residual tumor cells after co-culture of Vec-T, 19-22 CAR-T and 22-19 CAR-T with target cells for 24 hours at E:T ratio of 1:8, 1:4, 1:2 and 1:1, respectively, and a schematic flow diagram of percentages of residual tumor cells after co-culture for 48 hours at E:T ratio of 1:1 when the target cells were wild-type MV4-11 cell lines. FIG. 5I shows percentages of residual tumor cells after co-culture of vector-T, 19×22 CAR-T and 22×19 CAR-T with target cells for 24 hours at E:T ratio of 1:2, 1:1 and 2:1 when the target cells were MV4-11-CD19 and MV4-11-CD22 mixed at a ratio of 1:1. FIG. 5J shows percentages of residual tumor cells after co-culture of vector-T, 19×22 CAR-T and 22×19 CAR-T with target cells four 24 hours at E:T ratio of 1:2, 1:1 and 2:1, respectively, when the target cells were MV4-11. FIG. 5K shows percentages of residual tumor cells after co-culture of vector-T,1922-BB CAR-T, 1922-28B CAR-T, 1922-B28 CAR-Tand 1922-2828 CAR-T with target cells for 48 hours at E:T ratio of 1:1, 1:3 and 1:9, respectively, when the target cells were MV4-11-CD19 and MV4-11-CD22 mixed at a ratio of 1:1. FIG. 5L shows percentages of residual tumor cells after co-culture of vector-T, 1922-BB CAR-T, 1922-28B CAR-T, 1922-B28 CAR-T and 1922-2828 CAR-T with target cells for 48 hours at E:T ratio of 1:1, 1:3 and 1:9, respectively, when the target cells were MV4-11.

FIG. 6A and FIG. 6B show results of degranulation assays for a killing effect of vector-T and CAR-T on Namalwa, MV4-11-CD19, MV4-11-CD22 and MV4-11 (E:T ratio of 1:1) in an embodiment of the present invention, where CAR-T in FIG. 6A are 19 CAR-T, 22 CAR-T, 19-22 CAR-T and 22-19 CAR-T, respectively, and CAR-T in FIG. 6B are 19×22 CAR-T and 22×19 CAR-T, respectively.

FIG. 7A to FIG. 7C show results of levels of cytokines TNF-α (FIG. 7A) IL-2 (FIG. 7B) and IFN-γ(FIG. 7C) released from T cells after co-culture of vector-T, 19 CAR-T, 22 CAR-T, 19-22 CAR-T and 22-19 CAR-T with four target cells Namalwa, MV4-11-CD19, MV4-11-CD22 and MV4-11, respectively, for 48 hours at E:T ratio of 1:1 in an embodiment of the present invention.

FIG. 8 shows expression results of CD22 and CD19 target antigen molecules in bone marrow mononuclear cells (BMMCs) of B-ALL patients using flow cytometry, where P1 to P4 represent patient numbers.

FIG. 9 shows results of testing percentages of residual tumor cells by flow cytometry after co-culture of 22-19 CAR-T and 19-22 CAR-T with BMMCs from B-ALL patients at E:T ratio of 1:4 for 48 hours in an embodiment of the present invention.

FIG. 10 shows results of degranulation assays for a killing effect of 22-19 CAR-T and 19-22 CAR-T on BMMCs from B-ALL patients in an embodiment of the present invention.

FIG. 11A to FIG. 11D show evaluation results of an in vivo effect of 22-19 CAR-T and 19-22 CAR-T in xenograft mice in an embodiment of the present invention. Female NOD/SCID mice at 6-8 weeks were selected and randomly divided into two groups, and 5×10⁶ Namalwa cells were inoculated via tail vein on day 0, and 1×10⁷ vec-T cells or CAR-T cells (as shown in FIG. 11A) were administered intravenously on day 5, 9 and 12, respectively, and the mice were monitored weekly for weight change (as shown in FIG. 11B) after T cell injection. Tumor load plots are shown in FIG. 11C. Survival curves of the mice are shown in FIG. 11D. Survival time was calculated using SPSS software.

FIG. 12A and FIG. 12B show evaluation results of an in vivo effect of 22×19 CAR-T in mice in an embodiment of the present invention. Female NOD/SCID mice at 6-8 weeks were selected and randomly divided into two groups, 2.5×10⁶ Namalwa cells were inoculated via tail vein on day 0, and 1×10⁷ vec-T cells or CAR-T cells were administered intravenously on day 5, 9 and 12 (as shown in FIG. 12A), respectively. Survival curves of the mice are shown in FIG. 12B. Survival time was calculated using SPSS software.

### SEQUENCE DESCRIPTION

SEQ ID NO.1 is an amino acid sequence of CD19 scFv-CD22 scFv-CD8α-4-1 BB-CD3ζ of the present invention;

SEQ ID NO.2 is an amino acid sequence of CD22 scFv-CD19 scFv-CD8α-4-1 BB-CD3ζ of the present invention;

SEQ ID NO.3 is an amino acid sequence of CD19 VL-CD22 VL-CD22 VH-CD19 V_{H}-CD8α-4-1 BB-CD3ζ of the present invention;

SEQ ID NO.4 is an amino acid sequence of CD22 VL-CD19 VL-CD19 VH-CD22 VH-CD8α-4-1BB-CD3ζ of the present invention;

SEQ ID NO.5 is a nucleic acid sequence of CD19 scFv-CD22 scFv-CD8α-4-1 BB-CD3ζ of the present invention;

SEQ ID NO.6 is a nucleic acid sequence of CD22 scFv-CD19 scFv-CD8α-4-1 BB-CD3ζ of the present invention;

SEQ ID NO.7 is a nucleic acid sequence of CD19 VL-CD22 VL-CD22 VH-CD19 VH-CD8α-4-1BB-CD3ζ of the present invention;

SEQ ID NO.8 is a nucleic acid sequence of CD22 VL-CD19 VL-CD19 VH-CD22 VH-CD8α-4-1BB-CD3ζ of the present invention;

SEQ ID NO.9 is an amino acid sequence of a CD19 scFv-CD22 scFv-CD8α-4-1 BB-CD3ζ antigen recognition region of the present invention;

SEQ ID NO.10 is an amino acid sequence of a CD22 scFv-CD19 scFv-CD8α-4-1 BB-CD3ζ antigen recognition region of the present invention;

SEQ ID NO.11 is an amino acid sequence of a CD19 VL-CD22 VL-CD22 VH-CD19 VH-CD8α-4-1 BB-CD3ζ antigen recognition region of the present invention;

SEQ ID NO.12 is an amino acid sequence of a CD22 VL-CD19 VL-CD19 VH-CD22 VH-CD8α-4-1 BB-CD3ζ antigen recognition region of the present invention;

SEQ ID NO.13 is an amino acid sequence of a signal peptide in a chimeric antigen receptor targeting CD19 and CD22 of the present invention;

SEQ ID NO.14 is an amino acid sequence of CD8α-4-1BB-CD3ζ in a chimeric antigen receptor targeting CD19 and CD22 of the present invention;

SEQ ID NO.15 is an amino acid sequence of CD19 scFv-CD8α-4-1BB-CD3ζ-T2A-CD22 scFv-CD8α-4-1 BB-CD3ζ of the present invention;

SEQ ID NO.16 is an amino acid sequence of CD19 scFv-CD8α-CD28-CD3ζ-T2A-CD22 scFv-CD8α-4-1 BB-CD3ζ of the present invention;

SEQ ID NO.17 is an amino acid sequence of CD19 scFv-CD8α-4-1BB-CD3ζ-T2A-CD22 scFv-CD8α-CD28-CD3ζ of the present invention;

SEQ ID NO.18 is an amino acid sequence of CD19 scFv-CD8α-28-CD3ζ-T2A-CD22 scFv-CD8α-28-CD3ζ of the present invention;

SEQ ID NO.19 is a nucleic acid sequence of CD19 scFv-CD8α-4-1BB-CD3ζ-T2A-CD22 scFv-CD8α-4-1 BB-CD3ζ of the present invention;

SEQ ID NO.20 is a nucleic acid sequence of CD19 scFv-CD8α-CD28-CD3ζ-T2A-CD22 scFv-CD8α-4-1 BB-CD3ζ of the present invention;

SEQ ID NO.21 is a nucleic acid sequence of CD19 scFv-CD8α-4-1 BB-CD3ζ-T2A-CD22 scFv-CD8α-CD28-CD3ζ of the present invention;

SEQ ID NO.22 is a nucleic acid sequence of CD19 scFv-CD8α-CD28-CD3ζ-T2A-CD22 scFv-CD8α-CD28-CD3ζ of the present invention;

SEQ ID NO.23 is an amino acid sequence of a CD19 scFv-CD8α-4-1 BB-CD3ζ antigen recognition region of the present invention;

SEQ ID NO.24 is an amino acid sequence of a CD22 scFv-CD8α-CD28-CD3ζ antigen recognition region of the present invention;

SEQ ID NO.25 is an amino acid sequence of CD8α-CD28-CD3ζ in a chimeric antigen receptor targeting CD19 and CD22 of the present invention; and

SEQ ID NO. 26 is an amino acid sequence of T2A in a dual-target chimeric antigen receptor targeting CD19 and CD22 in the present invention.

SEQUENCE LIST

| | |
|---|---|
| Number | Sequence |
| SEQ ID NO.1 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQIYPG DGDTNYNGKFKGQATLTADKSSSTAYMQLSGLTSEDSAVYFCAR KTISSVVDFYFDYWGQGTTLTVSSEAAAKEAAAKEAAAKEAAAKE AAAKDIELTQSPSSLAVSVGEKVTMSCKSSQSLLYSSNQKNYLAW |
| | YQQKPGQSPKLLIYWASTRESGVPDRFTGSGSGTDFTLTVSSVKA EDLAVYYCQQSYSYPFTFGSGTKLEIKRGGGGSGGGGSGGGGS QVKLQQSGPELVKPGASVKISCKASGYDFSISWMNWVRQRPGQG LEWIGRIYPGDGDSNYNGKFEGKATLTADKSSSTAYMQLSGLTSV DSAVYFCARTTTMIALYAMDYWGQGTTVTVSSEFTTTPAPRPPTP APTIASQPLSLRPEACRPAAGGAVHTRGLDFACDIYIWAPLAGTCG VLLLSLVITLYKRGRKKLLYIFKQPFMRPVQTTQEEDGCSCRFPEE EEGGCELRVKFSRSADAPAYQQGQNQLYNELNLGRREEYDVLDK RRGRDPEMGGKPRRKNPQEGLYNELQKDKMAEAYSEIGMKGER RRGKGHDGLYQGLSTATKDTYDALHMQALPPR |
| SEQ ID NO.2 | DIELTQSPSSLAVSVGEKVTMSCKSSQSLLYSSNQKNYLAWYQQK PGQSPKLLlYWASTRESGVPDRFTGSGSGTDFTLTVSSVKAEDLA VYYCQQSYSYPFTFGSGTKLEIKRGGGGSGGGGSGGGGSQVKL QQSGPELVKPGASVKISCKASGYDFSISWMNWVRQRPGQGLEWI GRIYPGDGDSNYNGKFEGKATLTADKSSSTAYMQLSGLTSVDSAV YFCARTTTMIALYAMDYWGQGTTVTVSSEAAAKEAAAKEAAAKEA AAKEAAAKDIVLTQSPKFMSTSVGDRVSVTCKASQNVGTNVAWY QQKPGQSPKPLIYSATYRNSGVPDRFTGSGSGTDFTLTITNVQSK DLADYFCQQYNRYPYTSGGGTKLEIKRGGGGSGGGGSGGGGSQ VQLQQSGAELVRPGSSVKISCKASGYAFSSYWMNWVKQRPGQG LEWIGQIYPGDGDTNYNGKFKGQATLTADKSSSTAYMQLSGLTSE DSAVYFCARKTISSVVDFYFDYWGQGTTLTVSSEFTTTPAPRPPTP APTIASQPLSLRPEACRPAAGGAVHTRGLDFACDIYIWAPLAGTCG VLLLSLVITLYKRGRKKLLYIFKQPFMRPVQTTQEEDGCSCRFPEE EEGGCELRVKFSRSADAPAYQQGQNQLYNELNLGRREEYDVLDK RRGRDPEMGGKPRRKNPQEGLYNELQKDKMAEAYSEIGMKGER RRGKGHDGLYQGLSTATKDTYDALHMQALPPR |
| SEQ ID NO.3 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSDIELTQSPSSLAVSVGEKVTMS CKSSQSLLYSSNQKNYLAWYQQKPGQSPKLLIYWASTRESGVPD RFTGSGSGTDFTLTVSSVKAEDLAVYYCQQSYSYPFTFGSGTKLEI KRGSTSGSGKPGSGEGSTKGQVKLQQSGPELVKPGASVKISCKA |
| | SGYDFSISWMNWVRQRPGQGLEWIGRIYPGDGDSNYNGKFEGK ATLTADKSSSTAYMQLSGLTSVDSAVYFCARTTTMIALYAMDYWG QGTTVTVSSGGGGSQVQLQQSGAELVRPGSSVKISCKASGYAFS SYWMNWVKQRPGQGLEWIGQIYPGDGDTNYNGKFKGQATLTAD KSSSTAYMQLSGLTSEDSAVYFCARKTISSVVDFYFDYWGQGTTL TVSSEFTTTPAPRPPTPAPTIASQPLSLRPEACRPAAGGAVHTRGL DFACDIYIWAPLAGTCGVLLLSLVITLYKRGRKKLLYIFKQPFMRPV QTTQEEDGCSCRFPEEEEGGCELRVKFSRSADAPAYQQGQNQL YNELNLGRREEYDVLDKRRGRDPEMGGKPRRKNPQEGLYNELQ KDKMAEAYSEIGMKGERRRGKGHDGLYQGLSTATKDTYDALHMQ ALPPR |
| SEQ ID NO.4 | DIELTQSPSSLAVSVGEKVTMSCKSSQSLLYSSNQKNYLAWYQQK PGQSPKLLlYWASTRESGVPDRFTGSGSGTDFTLTVSSVKAEDLA VYYCQQSYSYPFTFGSGTKLEIKRGGGGSDIVLTQSPKFMSTSVG DRVSVTCKASQNVGTNVAWYQQKPGQSPKPLIYSATYRNSGVPD RFTGSGSGTDFTLTITNVQSKDLADYFCQQYNRYPYTSGGGTKLEI KRGSTSGSGKPGSGEGSTKGQVQLQQSGAELVRPGSSVKISCKA SGYAFSSYWMNWVKQRPGQGLEWIGQIYPGDGDTNYNGKFKGQ ATLTADKSSSTAYMQLSGLTSEDSAVYFCARKTISSVVDFYFDYW GQGTTLTVSSGGGGSQVKLQQSGPELVKPGASVKISCKASGYDF SISWMNWVRQRPGQGLEWIGRIYPGDGDSNYNGKFEGKATLTAD KSSSTAYMQLSGLTSVDSAVYFCARTTTMIALYAMDYWGQGTTVT VSSEFTTTPAPRPPTPAPTIASQPLSLRPEACRPAAGGAVHTRGLD FACDIYIWAPLAGTCGVLLLSLVITLYKRGRKKLLYIFKQPFMRPVQ TTQEEDGCSCRFPEEEEGGCELRVKFSRSADAPAYQQGQNQLYN ELNLGRREEYDVLDKRRGRDPEMGGKPRRKNPQEGLYNELQKD KMAEAYSEIGMKGERRRGKGHDGLYQGLSTATKDTYDALHMQAL PPR |
| SEQ ID NO.5 | gatattgtgctgacccagagccccaagttcatgagcaccagcgtgggcgatagagtgagcgt gacctgcaaggcaagccagaacgtgggaacaaacgtggcctggtaccaacagaaacccg gccaaagccctaagcccctgatttacagcgccacctacagaaatagcggcgtgcccgacag atttacaggaagcggcagcggaaccgatttcacactgaccatcaccaacgtgcagagcaaa gacctggccgactacttctgccagcagtacaacagatacccctacaccagcggaggagga |
| | acaaagctggagatcaagagaggtggtggtggttctggcggcggcggctccggtggtggtg gttctcaagtgcaactgcaacagagcggagccgaactggtgagacccggaagcagcgtga agatcagctgcaaggcttccggctacgcctttagcagctactggatgaactgggtgaagcag agacctggacagggactggaatggatcggccagatttaccctggagacggcgacacaaac tacaacggcaagttcaagggccaagctacactgaccgccgacaaaagcagcagcaccgc ctatatgcagctgagcggactgaccagcgaagatagcgctgtgtacttctgcgccagaaaga ccatcagcagcgtggtggacttctacttcgactactggggacaaggcaccaccctgacagtg agcagcgaagccgctgctaaggaagccgctgctaaggaagccgctgctaaggaagccgct gctaaggaagccgctgctaaggacattgagctcacccagtctccatcctccctagctgtgtca gttggagagaaggttactatgagctgcaagtccagtcagagccttttatatagtagcaatcaaa agaactatttggcctggtaccagcagaaaccagggcagtctcctaaactgctgatttactggg catccactagggaatctggggtccctgatcgcttcacaggcagtggatctgggacagatttcac tctcaccgtcagcagtgtgaaggctgaagacctggcagtttattactgtcagcaatcttatagtta tccattcacattcggctcgggcaccaagctggaaatcaaacggggtggtggtggttctggcgg cggcggctccggtggtggtggttctcaggtcaaactgcagcagtcaggacctgaactggtga agcctggggcctcagtgaagatttcctgcaaagcttctggctacgatttcagtatttcttggatga actgggtgaggcagaggcctggacagggtcttgagtggattggacggatttatcctggagatg gagatagtaactacaatgggaagttcgagggcaaggccacactgactgcagacaaatcctc cagcacagcctacatgcagctcagcggcctgacctctgtggactctgcggtctatttttgtgcaa gaaccaccactatgattgccctctatgctatggactactggggccaagggaccacggtcacc gtctcctcagaattcaccacgacgccagcgccgcgaccaccaacaccggcgcccaccatc gcgtcgcagcccctgtccctgcgcccagaggcgtgccggccagcggcggggggcgcagtg cacacgagggggctggacttcgcctgtgatatctacatctgggcgcccttggccgggacttgtg gggtccttctcctgtcactggttatcaccctttacaaacggggcagaaagaaactcctgtatatat tcaaacaaccatttatgagaccagtacaaactactcaagaggaagatggctgtagctgccga tttccagaagaagaagaaggaggatgtgaactgagagtgaagttcagcaggagcgcagac gcccccgcgtaccagcagggccagaaccagctctataacgagctcaatctaggacgaaga gaggagtacgatgttttggacaagagacgtggccgggaccctgagatggggggaaagccg agaaggaagaaccctcaggaaggcctgtacaatgaactgcagaaagataagatggcgga ggcctacagtgagattgggatgaaaggcgagcgccggaggggcaaggggcacgatggcc tttaccagggtctcagtacagccaccaaggacacctacgacgcccttcacatgcaggccctg ccccctcgc |
| SEQ ID | gacattgagctcacccagtctccatcctccctagctgtgtcagttggagagaaggttactatgag |
| NO.6 | ctgcaagtccagtcagagccttttatatagtagcaatcaaaagaactatttggcctggtaccag cagaaaccagggcagtctcctaaactgctgatttactgggcatccactagggaatctggggtc cctgatcgcttcacaggcagtggatctgggacagatttcactctcaccgtcagcagtgtgaagg ctgaagacctggcagtttattactgtcagcaatcttatagttatccattcacattcggctcgggcac caagctggaaatcaaacggggtggtggtggttctggcggcggcggctccggtggtggtggttc tcaggtcaaactgcagcagtcaggacctgaactggtgaagcctggggcctcagtgaagattt cctgcaaagcttctggctacgatttcagtatttcttggatgaactgggtgaggcagaggcctgga cagggtcttgagtggattggacggatttatcctggagatggagatagtaactacaatgggaagt tcgagggcaaggccacactgactgcagacaaatcctccagcacagcctacatgcagctca gcggcctgacctctgtggactctgcggtctatttttgtgcaagaaccaccactatgattgccctct atgctatggactactggggccaagggaccacggtcaccgtctcctcagaagccgctgctaag gaagccgctgctaaggaagccgctgctaaggaagccgctgctaaggaagccgctgctaag gatattgtgctgacccagagccccaagttcatgagcaccagcgtgggcgatagagtgagcgt gacctgcaaggcaagccagaacgtgggaacaaacgtggcctggtaccaacagaaacccg gccaaagccctaagcccctgatttacagcgccacctacagaaatagcggcgtgcccgacag atttacaggaagcggcagcggaaccgatttcacactgaccatcaccaacgtgcagagcaaa gacctggccgactacttctgccagcagtacaacagatacccctacaccagcggaggagga acaaagctggagatcaagagaggtggtggtggttctggcggcggcggctccggtggtggtg gttctcaagtgcaactgcaacagagcggagccgaactggtgagacccggaagcagcgtga agatcagctgcaaggcttccggctacgcctttagcagctactggatgaactgggtgaagcag agacctggacagggactggaatggatcggccagatttaccctggagacggcgacacaaac tacaacggcaagttcaagggccaagctacactgaccgccgacaaaagcagcagcaccgc ctatatgcagctgagcggactgaccagcgaagatagcgctgtgtacttctgcgccagaaaga ccatcagcagcgtggtggacttctacttcgactactggggacaaggcaccaccctgacagtg agcagcgaattcaccacgacgccagcgccgcgaccaccaacaccggcgcccaccatcgc gtcgcagcccctgtccctgcgcccagaggcgtgccggccagcggcggggggcgcagtgca cacgagggggctggacttcgcctgtgatatctacatctgggcgcccttggccgggacttgtggg gtccttctcctgtcactggttatcaccctttacaaacggggcagaaagaaactcctgtatatattc aaacaaccatttatgagaccagtacaaactactcaagaggaagatggctgtagctgccgattt ccagaagaagaagaaggaggatgtgaactgagagtgaagttcagcaggagcgcagacg cccccgcgtaccagcagggccagaaccagctctataacgagctcaatctaggacgaagag aggagtacgatgttttggacaagagacgtggccgggaccctgagatggggggaaagccga gaaggaagaaccctcaggaaggcctgtacaatgaactgcagaaagataagatggcggag |
| | gcctacagtgagattgggatgaaaggcgagcgccggaggggcaaggggcacgatggcctt taccagggtctcagtacagccaccaaggacacctacgacgcccttcacatgcaggccctgc cccctcgc |
| SEQ ID NO.7 | gatattgtgctgacccagagccccaagttcatgagcaccagcgtgggcgatagagtgagcgt gacctgcaaggcaagccagaacgtgggaacaaacgtggcctggtaccaacagaaacccg gccaaagccctaagcccctgatttacagcgccacctacagaaatagcggcgtgcccgacag atttacaggaagcggcagcggaaccgatttcacactgaccatcaccaacgtgcagagcaaa gacctggccgactacttctgccagcagtacaacagatacccctacaccagcggaggagga acaaagctggagatcaagagaggcggcggaggttctgacattgagctcacccagtctccat cctccctagctgtgtcagttggagagaaggttactatgagctgcaagtccagtcagagcctttta tatagtagcaatcaaaagaactatttggcctggtaccagcagaaaccagggcagtctcctaa actgctgatttactgggcatccactagggaatctggggtccctgatcgcttcacaggcagtgga tctgggacagatttcactctcaccgtcagcagtgtgaaggctgaagacctggcagtttattactg tcagcaatcttatagttatccattcacattcggctcgggcaccaagctggaaatcaaacggggc agcacaagcggctctggcaagcctggatctggcgagggctctaccaagggccaggtcaaa ctgcagcagtcaggacctgaactggtgaagcctggggcctcagtgaagatttcctgcaaagc ttctggctacgatttcagtatttcttggatgaactgggtgaggcagaggcctggacagggtcttg agtggattggacggatttatcctggagatggagatagtaactacaatgggaagttcgagggca aggccacactgactgcagacaaatcctccagcacagcctacatgcagctcagcggcctgac ctctgtggactctgcggtctatttttgtgcaagaaccaccactatgattgccctctatgctatggact actggggccaagggaccacggtcaccgtctcctcaggcggcggaggttctcaagtgcaact gcaacagagcggagccgaactggtgagacccggaagcagcgtgaagatcagctgcaag gcttccggctacgcctttagcagctactggatgaactgggtgaagcagagacctggacaggg actggaatggatcggccagatttaccctggagacggcgacacaaactacaacggcaagttc aagggccaagctacactgaccgccgacaaaagcagcagcaccgcctatatgcagctgag cggactgaccagcgaagatagcgctgtgtacttctgcgccagaaagaccatcagcagcgtg gtggacttctacttcgactactggggacaaggcaccaccctgacagtgagcagcgaattcac cacgacgccagcgccgcgaccaccaacaccggcgcccaccatcgcgtcgcagcccctgt ccctgcgcccagaggcgtgccggccagcggcggggggcgcagtgcacacgagggggct ggacttcgcctgtgatatctacatctgggcgcccttggccgggacttgtggggtccttctcctgtc actggttatcaccctttacaaacggggcagaaagaaactcctgtatatattcaaacaaccattt atgagaccagtacaaactactcaagaggaagatggctgtagctgccgatttccagaagaag aagaaggaggatgtgaactgagagtgaagttcagcaggagcgcagacgcccccgcgtac |
| | cagcagggccagaaccagctctataacgagctcaatctaggacgaagagaggagtacgat gttttggacaagagacgtggccgggaccctgagatggggggaaagccgagaaggaagaa ccctcaggaaggcctgtacaatgaactgcagaaagataagatggcggaggcctacagtga gattgggatgaaaggcgagcgccggaggggcaaggggcacgatggcctttaccagggtct cagtacagccaccaaggacacctacgacgcccttcacatgcaggccctgccccctcgc |
| SEQ ID NO.8 | gacattgagctcacccagtctccatcctccctagctgtgtcagttggagagaaggttactatgag ctgcaagtccagtcagagccttttatatagtagcaatcaaaagaactatttggcctggtaccag cagaaaccagggcagtctcctaaactgctgatttactgggcatccactagggaatctggggtc cctgatcgcttcacaggcagtggatctgggacagatttcactctcaccgtcagcagtgtgaagg ctgaagacctggcagtttattactgtcagcaatcttatagttatccattcacattcggctcgggcac caagctggaaatcaaacggggcggcggaggttctgatattgtgctgacccagagccccaag ttcatgagcaccagcgtgggcgatagagtgagcgtgacctgcaaggcaagccagaacgtg ggaacaaacgtggcctggtaccaacagaaacccggccaaagccctaagcccctgatttac agcgccacctacagaaatagcggcgtgcccgacagatttacaggaagcggcagcggaac cgatttcacactgaccatcaccaacgtgcagagcaaagacctggccgactacttctgccagc agtacaacagatacccctacaccagcggaggaggaacaaagctggagatcaagagagg cagcacaagcggctctggcaagcctggatctggcgagggctctaccaagggccaagtgca actgcaacagagcggagccgaactggtgagacccggaagcagcgtgaagatcagctgca aggcttccggctacgcctttagcagctactggatgaactgggtgaagcagagacctggacag ggactggaatggatcggccagatttaccctggagacggcgacacaaactacaacggcaag ttcaagggccaagctacactgaccgccgacaaaagcagcagcaccgcctatatgcagctg agcggactgaccagcgaagatagcgctgtgtacttctgcgccagaaagaccatcagcagcg tggtggacttctacttcgactactggggacaaggcaccaccctgacagtgagcagcggcggc ggaggttctcaggtcaaactgcagcagtcaggacctgaactggtgaagcctggggcctcagt gaagatttcctgcaaagcttctggctacgatttcagtatttcttggatgaactgggtgaggcaga ggcctggacagggtcttgagtggattggacggatttatcctggagatggagatagtaactaca atgggaagttcgagggcaaggccacactgactgcagacaaatcctccagcacagcctacat gcagctcagcggcctgacctctgtggactctgcggtctatttttgtgcaagaaccaccactatga ttgccctctatgctatggactactggggccaagggaccacggtcaccgtctcctcagaattcac cacgacgccagcgccgcgaccaccaacaccggcgcccaccatcgcgtcgcagcccctgt ccctgcgcccagaggcgtgccggccagcggcggggggcgcagtgcacacgagggggct ggacttcgcctgtgatatctacatctgggcgcccttggccgggacttgtggggtccttctcctgtc actggttatcaccctttacaaacggggcagaaagaaactcctgtatatattcaaacaaccattt |
| | atgagaccagtacaaactactcaagaggaagatggctgtagctgccgatttccagaagaag aagaaggaggatgtgaactgagagtgaagttcagcaggagcgcagacgcccccgcgtac cagcagggccagaaccagctctataacgagctcaatctaggacgaagagaggagtacgat gttttggacaagagacgtggccgggaccctgagatggggggaaagccgagaaggaagaa ccctcaggaaggcctgtacaatgaactgcagaaagataagatggcggaggcctacagtga gattgggatgaaaggcgagcgccggaggggcaaggggcacgatggcctttaccagggtct cagtacagccaccaaggacacctacgacgcccttcacatgcaggccctgccccctcgc |
| SEQ ID NO.9 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQIYPG DGDTNYNGKFKGQATLTADKSSSTAYMQLSGLTSEDSAVYFCAR KTISSVVDFYFDYWGQGTTLTVSSEAAAKEAAAKEAAAKEAAAKE AAAKDIELTQSPSSLAVSVGEKVTMSCKSSQSLLYSSNQKNYLAW YQQKPGQSPKLLIYWASTRESGVPDRFTGSGSGTDFTLTVSSVKA EDLAVYYCQQSYSYPFTFGSGTKLEIKRGGGGSGGGGSGGGGS QVKLQQSGPELVKPGASVKISCKASGYDFSISWMNWVRQRPGQG LEWIGRIYPGDGDSNYNGKFEGKATLTADKSSSTAYMQLSGLTSV DSAVYFCARTTTMIALYAMDYWGQGTTVTVSS |
| SEQ ID NO.10 | DIELTQSPSSLAVSVGEKVTMSCKSSQSLLYSSNQKNYLAWYQQK PGQSPKLLIYWASTRESGVPDRFTGSGSGTDFTLTVSSVKAEDLA VYYCQQSYSYPFTFGSGTKLEIKRGGGGSGGGGSGGGGSQVKL QQSGPELVKPGASVKISCKASGYDFSISWMNWVRQRPGQGLEWI GRIYPGDGDSNYNGKFEGKATLTADKSSSTAYMQLSGLTSVDSAV YFCARTTTMIALYAMDYWGQGTTVTVSSEAAAKEAAAKEAAAKEA AAKEAAAKDIVLTQSPKFMSTSVGDRVSVTCKASQNVGTNVAWY QQKPGQSPKPLIYSATYRNSGVPDRFTGSGSGTDFTLTITNVQSK DLADYFCQQYNRYPYTSGGGTKLEIKRGGGGSGGGGSGGGGSQ VQLQQSGAELVRPGSSVKISCKASGYAFSSYWMNWVKQRPGQG LEWIGQIYPGDGDTNYNGKFKGQATLTADKSSSTAYMQLSGLTSE DSAVYFCARKTISSVVDFYFDYWGQGTTLTVSS |
| SEQ ID NO.11 | DIVLTQSPKFMSTSVGDRVSVTCKASQNVGTNVAWYQQKPGQSP KPLIYSATYRNSGVPDRFTGSGSGTDFTLTITNVQSKDLADYFCQQ |
| | YNRYPYTSGGGTKLEIKRGGGGSDIELTQSPSSLAVSVGEKVTMS CKSSQSLLYSSNQKNYLAWYQQKPGQSPKLLIYWASTRESGVPD RFTGSGSGTDFTLTVSSVKAEDLAVYYCQQSYSYPFTFGSGTKLEI KRGSTSGSGKPGSGEGSTKGQVKLQQSGPELVKPGASVKISCKA SGYDFSISWMNWVRQRPGQGLEWIGRIYPGDGDSNYNGKFEGK ATLTADKSSSTAYMQLSGLTSVDSAVYFCARTTTMIALYAMDYWG QGTTVTVSSGGGGSQVQLQQSGAELVRPGSSVKISCKASGYAFS SYWMNWVKQRPGQGLEWIGQIYPGDGDTNYNGKFKGQATLTAD KSSSTAYMQLSGLTSEDSAVYFCARKTISSVVDFYFDYWGQGTTL TVSS |
| SEQ ID NO.12 | DIELTQSPSSLAVSVGEKVTMSCKSSQSLLYSSNQKNYLAWYQQK PGQSPKLLlYWASTRESGVPDRFTGSGSGTDFTLTVSSVKAEDLA VYYCQQSYSYPFTFGSGTKLEIKRGGGGSDIVLTQSPKFMSTSVG DRVSVTCKASQNVGTNVAWYQQKPGQSPKPLIYSATYRNSGVPD RFTGSGSGTDFTLTITNVQSKDLADYFCQQYNRYPYTSGGGTKLEl KRGSTSGSGKPGSGEGSTKGQVQLQQSGAELVRPGSSVKISCKA SGYAFSSYWMNWVKQRPGQGLEWIGQIYPGDGDTNYNGKFKGQ ATLTADKSSSTAYMQLSGLTSEDSAVYFCARKTISSVVDFYFDYW GQGTTLTVSSGGGGSQVKLQQSGPELVKPGASVKISCKASGYDF SISWMNWVRQRPGQGLEWIGRIYPGDGDSNYNGKFEGKATLTAD KSSSTAYMQLSGLTSVDSAVYFCARTTTMIALYAMDYWGQGTTVT VSS |
| SEQ ID NO.13 | MALPVTALLLPLALLLHAARP |
| SEQ ID NO.14 | TTTPAPRPPTPAPTIASQPLSLRPEACRPAAGGAVHTRGLDFACDI YIWAPLAGTCGVLLLSLVITLYKRGRKKLLYIFKQPFMRPVQTTQEE DGCSCRFPEEEEGGCELRVKFSRSADAPAYQQGQNQLYNELNLG RREEYDVLDKRRGRDPEMGGKPRRKNPQEGLYNELQKDKMAEA YSEIGMKGERRRGKGHDGLYQGLSTATKDTYDALHMQALPPR |
| SEQ ID NO.15 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQlYPG |
| | DGDTNYNGKFKGQATLTADKSSSTAYMQLSGLTSEDSAVYFCAR KTISSVVDFYFDYWGQGTTLTVSSFETTTPAPRPPTPAPTIASQPL SLRPEACRPAAGGAVHTRGLDFACDIYIWAPLAGTCGVLLLSLVITL YCKRGRKKLLYlFKQPFMRPVQTTQEEDGCSCRFPEEEEGGCEL RVKFSRSADAPAYQQGQNQLYNELNLGRREEYDVLDKRRGRDPE MGGKPRRKNPQEGLYNELQKDKMAEAYSEIGMKGERRRGKGHD GLYQGLSTATKDTYDALHMQALPPRAAAEGRGSLLTCGDVEENP GPSGATMALPVTALLLPLALLLHAARPGSDIELTQSPSSLAVSVGE KVTMSCKSSQSLLYSSNQKNYLAWYQQKPGQSPKLLIYWASTRE SGVPDRFTGSGSGTDFTLTVSSVKAEDLAVYYCQQSYSYPFTFGS GTKLEIKRGGGGSGGGGSGGGGSQVKLQQSGPELVKPGASVKIS CKASGYDFSISWMNWVRQRPGQGLEWIGRIYPGDGDSNYNGKF EGKATLTADKSSSTAYMQLSGLTSVDSAVYFCARTTTMIALYAMD YWGQGTTVTVSSEFTTTPAPRPPTPAPTIASQPLSLRPEACRPAA GGAVHTRGLDFACDIYIWAPLAGTCGVLLLSLVITLYCKRGRKKLLY IFKQPFMRPVQTTQEEDGCSCRFPEEEEGGCELRVKFSRSADAP AYQQGQNQLYNELNLGRREEYDVLDKRRGRDPEMGGKPRRKNP QEGLYNELQKDKMAEAYSEIGMKGERRRGKGHDGLYQGLSTATK DTYDALHMQALPPR |
| SEQ ID NO.16 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQIYPG DGDTNYNGKFKGQATLTADKSSSTAYMQLSGLTSEDSAVYFCAR KTISSVVDFYFDYWGQGTTLTVSSFETTTPAPRPPTPAPTIASQPL SLRPEACRPAAGGAVHTRGLDFACDIYIWAPLAGTCGVLLLSLVITL YCRSKRSRLLHSDYMNMTPRRPGPTRKHYQPYAPPRDFAAYRSR VKFSRSADAPAYQQGQNQLYNELNLGRREEYDVLDKRRGRDPE MGGKPRRKNPQEGLYNELQKDKMAEAYSEIGMKGERRRGKGHD GLYQGLSTATKDTYDALHMQALPPRAAAEGRGSLLTCGDVEENP GPSGATMALPVTALLLPLALLLHAARPGSDIELTQSPSSLAVSVGE KVTMSCKSSQSLLYSSNQKNYLAWYQQKPGQSPKLLIYWASTRE SGVPDRFTGSGSGTDFTLTVSSVKAEDLAVYYCQQSYSYPFTFGS |
| | GTKLEIKRGGGGSGGGGSGGGGSQVKLQQSGPELVKPGASVKIS CKASGYDFSISWMNWVRQRPGQGLEWIGRIYPGDGDSNYNGKF EGKATLTADKSSSTAYMQLSGLTSVDSAVYFCARTTTMIALYAMD YWGQGTTVTVSSEFTTTPAPRPPTPAPTIASQPLSLRPEACRPAA GGAVHTRGLDFACDIYIWAPLAGTCGVLLLSLVITLYCKRGRKKLLY IFKQPFMRPVQTTQEEDGCSCRFPEEEEGGCELRVKFSRSADAP AYQQGQNQLYNELNLGRREEYDVLDKRRGRDPEMGGKPRRKNP QEGLYNELQKDKMAEAYSEIGMKGERRRGKGHDGLYQGLSTATK DTYDALHMQALPPR |
| SEQ ID NO.17 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQIYPG DGDTNYNGKFKGQATLTADKSSSTAYMQLSGLTSEDSAVYFCAR KTISSVVDFYFDYWGQGTTLTVSSFETTTPAPRPPTPAPTIASQPL SLRPEACRPAAGGAVHTRGLDFACDIYIWAPLAGTCGVLLLSLVITL YCKRGRKKLLYIFKQPFMRPVQTTQEEDGCSCRFPEEEEGGCEL RVKFSRSADAPAYQQGQNQLYNELNLGRREEYDVLDKRRGRDPE MGGKPRRKNPQEGLYNELQKDKMAEAYSEIGMKGERRRGKGHD GLYQGLSTATKDTYDALHMQALPPRAAAEGRGSLLTCGDVEENP GPSGATMALPVTALLLPLALLLHAARPGSDIELTQSPSSLAVSVGE KVTMSCKSSQSLLYSSNQKNYLAWYQQKPGQSPKLLIYWASTRE SGVPDRFTGSGSGTDFTLTVSSVKAEDLAVYYCQQSYSYPFTFGS GTKLEIKRGGGGSGGGGSGGGGSQVKLQQSGPELVKPGASVKIS CKASGYDFSISWMNWVRQRPGQGLEWIGRIYPGDGDSNYNGKF EGKATLTADKSSSTAYMQLSGLTSVDSAVYFCARTTTMIALYAMD YWGQGTTVTVSSEFTTTPAPRPPTPAPTIASQPLSLRPEACRPAA GGAVHTRGLDFACDIYIWAPLAGTCGVLLLSLVITLYCRSKRSRLLH SDYMNMTPRRPGPTRKHYQPYAPPRDFAAYRSRVKFSRSADAPA YQQGQNQLYNELNLGRREEYDVLDKRRGRDPEMGGKPRRKNPQ EGLYNELQKDKMAEAYSEIGMKGERRRGKGHDGLYQGLSTATKD TYDALHMQALPPR |
| SEQ ID | DIVLTQSPKFMSTSVGDRVSVTCKASQNVGTNVAWYQQKPGQSP |
| NO.18 | KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQIYPG DGDTNYNGKFKGQATLTADKSSSTAYMQLSGLTSEDSAVYFCAR KTISSVVDFYFDYWGQGTTLTVSSFETTTPAPRPPTPAPTIASQPL SLRPEACRPAAGGAVHTRGLDFACDIYIWAPLAGTCGVLLLSLVITL YCRSKRSRLLHSDYMNMTPRRPGPTRKHYQPYAPPRDFAAYRSR VKFSRSADAPAYQQGQNQLYNELNLGRREEYDVLDKRRGRDPE MGGKPRRKNPQEGLYNELQKDKMAEAYSEIGMKGERRRGKGHD GLYQGLSTATKDTYDALHMQALPPRAAAEGRGSLLTCGDVEENP GPSGATMALPVTALLLPLALLLHAARPGSDIELTQSPSSLAVSVGE KVTMSCKSSQSLLYSSNQKNYLAWYQQKPGQSPKLLIYWASTRE SGVPDRFTGSGSGTDFTLTVSSVKAEDLAVYYCQQSYSYPFTFGS GTKLEIKRGGGGSGGGGSGGGGSQVKLQQSGPELVKPGASVKIS CKASGYDFSISWMNWVRQRPGQGLEWIGRIYPGDGDSNYNGKF EGKATLTADKSSSTAYMQLSGLTSVDSAVYFCARTTTMIALYAMD YWGQGTTVTVSSEFTTTPAPRPPTPAPTIASQPLSLRPEACRPAA GGAVHTRGLDFACDIYIWAPLAGTCGVLLLSLVITLYCRSKRSRLLH SDYMNMTPRRPGPTRKHYQPYAPPRDFAAYRSRVKFSRSADAPA YQQGQNQLYNELNLGRREEYDVLDKRRGRDPEMGGKPRRKNPQ EGLYNELQKDKMAEAYSEIGMKGERRRGKGHDGLYQGLSTATKD TYDALHMQALPPR |
| SEQ ID NO.19 | gatattgtgctgacccagagccccaagttcatgagcaccagcgtgggcgatagagtgagcgt gacctgcaaggcaagccagaacgtgggaacaaacgtggcctggtaccaacagaaacccg gccaaagccctaagcccctgatttacagcgccacctacagaaatagcggcgtgcccgacag atttacaggaagcggcagcggaaccgatttcacactgaccatcaccaacgtgcagagcaaa gacctggccgactacttctgccagcagtacaacagatacccctacaccagcggaggagga acaaagctggagatcaagagaggtggtggtggttctggcggcggcggctccggtggtggtg gttctcaagtgcaactgcaacagagcggagccgaactggtgagacccggaagcagcgtga agatcagctgcaaggcttccggctacgcctttagcagctactggatgaactgggtgaagcag agacctggacagggactggaatggatcggccagatttaccctggagacggcgacacaaac tacaacggcaagttcaagggccaagctacactgaccgccgacaaaagcagcagcaccgc |
| | ctatatgcagctgagcggactgaccagcgaagatagcgctgtgtacttctgcgccagaaaga ccatcagcagcgtggtggacttctacttcgactactggggacaaggcaccaccctgacagtg agcagcttcgaaaccacgacgccagcgccgcgaccaccaacaccggcgcccaccatcgc gtcgcagcccctgtccctgcgcccagaggcgtgccggccagcggcggggggcgcagtgca cacgagggggctggacttcgcctgtgatatctacatctgggcgcccttggccgggacttgtggg gtccttctcctgtcactggttatcaccctttactgcaaacggggcagaaagaaactcctgtatata ttcaaacaaccatttatgagaccagtacaaactactcaagaggaagatggctgtagctgccg atttccagaagaagaagaaggaggatgtgaactgagagtgaagttcagcaggagcgcaga cgcccccgcgtaccagcagggccagaaccagctctataacgagctcaatctaggacgaag agaggagtacgatgttttggacaagagacgtggccgggaccctgagatggggggaaagcc gagaaggaagaaccctcaggaaggcctgtacaatgaactgcagaaagataagatggcgg aggcctacagtgagattgggatgaaaggcgagcgccggaggggcaaggggcacgatgg cctttaccagggtctcagtacagccaccaaggacacctacgacgcccttcacatgcaggccc tgccccctcgcgcggccgctgagggcagaggaagtcttctaacatgcggtgacgtggagga gaatcccggcccttccggagccaccatggccttaccagtgaccgccttgctcctgccgctggc cttgctgctccacgccgccaggccgggatccgacattgagctcacccagtctccatcctcccta gctgtgtcagttggagagaaggttactatgagctgcaagtccagtcagagccttttatatagtag caatcaaaagaactatttggcctggtaccagcagaaaccagggcagtctcctaaactgctga tttactgggcatccactagggaatctggggtccctgatcgcttcacaggcagtggatctgggac agatttcactctcaccgtcagcagtgtgaaggctgaagacctggcagtttattactgtcagcaat cttatagttatccattcacattcggctcgggcaccaagctggaaatcaaacggggtggtggtgg ttctggcggcggcggctccggtggtggtggttctcaggtcaaactgcagcagtcaggacctga actggtgaagcctggggcctcagtgaagatttcctgcaaagcttctggctacgatttcagtatttc ttggatgaactgggtgaggcagaggcctggacagggtcttgagtggattggacggatttatcct ggagatggagatagtaactacaatgggaagttcgagggcaaggccacactgactgcagac aaatcctccagcacagcctacatgcagctcagcggcctgacctctgtggactctgcggtctattt ttgtgcaagaaccaccactatgattgccctctatgctatggactactggggccaagggaccac ggtcaccgtctcctcagaattcaccacgacgccagcgccgcgaccaccaacaccggcgcc caccatcgcgtcgcagcccctgtccctgcgcccagaggcgtgccggccagcggcgggggg cgcagtgcacacgagggggctggacttcgcctgtgatatctacatctgggcgcccttggccgg gacttgtggggtccttctcctgtcactggttatcaccctttactgcaaacggggcagaaagaaac |
| | tcctgtatatattcaaacaaccatttatgagaccagtacaaactactcaagaggaagatggctg tagctgccgatttccagaagaagaagaaggaggatgtgaactgagagtgaagttcagcagg agcgcagacgcccccgcgtaccagcagggccagaaccagctctataacgagctcaatcta ggacgaagagaggagtacgatgttttggacaagagacgtggccgggaccctgagatgggg ggaaagccgagaaggaagaaccctcaggaaggcctgtacaatgaactgcagaaagata agatggcggaggcctacagtgagattgggatgaaaggcgagcgccggaggggcaaggg gcacgatggcctttaccagggtctcagtacagccaccaaggacacctacgacgcccttcaca tgcaggccctgccccctcgctaa |
| SEQ ID NO.20 | gatattgtgctgacccagagccccaagttcatgagcaccagcgtgggcgatagagtgagcgt gacctgcaaggcaagccagaacgtgggaacaaacgtggcctggtaccaacagaaacccg gccaaagccctaagcccctgatttacagcgccacctacagaaatagcggcgtgcccgacag atttacaggaagcggcagcggaaccgatttcacactgaccatcaccaacgtgcagagcaaa gacctggccgactacttctgccagcagtacaacagatacccctacaccagcggaggagga acaaagctggagatcaagagaggtggtggtggttctggcggcggcggctccggtggtggtg gttctcaagtgcaactgcaacagagcggagccgaactggtgagacccggaagcagcgtga agatcagctgcaaggcttccggctacgcctttagcagctactggatgaactgggtgaagcag agacctggacagggactggaatggatcggccagatttaccctggagacggcgacacaaac tacaacggcaagttcaagggccaagctacactgaccgccgacaaaagcagcagcaccgc ctatatgcagctgagcggactgaccagcgaagatagcgctgtgtacttctgcgccagaaaga ccatcagcagcgtggtggacttctacttcgactactggggacaaggcaccaccctgacagtg agcagcttcgaaaccacgacgccagcgccgcgaccaccaacaccggcgcccaccatcgc gtcgcagcccctgtccctgcgcccagaggcgtgccggccagcggcggggggcgcagtgca cacgagggggctggacttcgcctgtgatatctacatctgggcgcccttggccgggacttgtggg gtccttctcctgtcactggttatcaccctttactgcaggagtaagaggagcaggctcctgcacag tgactacatgaacatgactccccgccgccccgggcccacccgcaagcattaccagccctatg ccccaccacgcgacttcgcagcctatcgctccagagtgaagttcagcaggagcgcagacgc ccccgcgtaccagcagggccagaaccagctctataacgagctcaatctaggacgaagaga ggagtacgatgttttggacaagagacgtggccgggaccctgagatggggggaaagccgag aaggaagaaccctcaggaaggcctgtacaatgaactgcagaaagataagatggcggagg cctacagtgagattgggatgaaaggcgagcgccggaggggcaaggggcacgatggccttt accagggtctcagtacagccaccaaggacacctacgacgcccttcacatgcaggccctgcc ccctcgcgcggccgctgagggcagaggaagtcttctaacatgcggtgacgtggaggagaat |
| | cccggcccttccggagccaccatggccttaccagtgaccgccttgctcctgccgctggccttgc tgctccacgccgccaggccgggatccgacattgagctcacccagtctccatcctccctagctgt gtcagttggagagaaggttactatgagctgcaagtccagtcagagccttttatatagtagcaat caaaagaactatttggcctggtaccagcagaaaccagggcagtctcctaaactgctgatttac tgggcatccactagggaatctggggtccctgatcgcttcacaggcagtggatctgggacagat ttcactctcaccgtcagcagtgtgaaggctgaagacctggcagtttattactgtcagcaatcttat agttatccattcacattcggctcgggcaccaagctggaaatcaaacggggtggtggtggttctg gcggcggcggctccggtggtggtggttctcaggtcaaactgcagcagtcaggacctgaactg gtgaagcctggggcctcagtgaagatttcctgcaaagcttctggctacgatttcagtatttcttgg atgaactgggtgaggcagaggcctggacagggtcttgagtggattggacggatttatcctgga gatggagatagtaactacaatgggaagttcgagggcaaggccacactgactgcagacaaa tcctccagcacagcctacatgcagctcagcggcctgacctctgtggactctgcggtctattt^{t}tgt gcaagaaccaccactatgattgccctctatgctatggactactggggccaagggaccacggt caccgtctcctcagaattcaccacgacgccagcgccgcgaccaccaacaccggcgcccac catcgcgtcgcagcccctgtccctgcgcccagaggcgtgccggccagcggcggggggcgc agtgcacacgagggggctggacttcgcctgtgatatctacatctgggcgcccttggccgggac ttgtggggtccttctcctgtcactggttatcaccctttactgcaaacggggcagaaagaaactcct gtatatattcaaacaaccatttatgagaccagtacaaactactcaagaggaagatggctgtag ctgccgatttccagaagaagaagaaggaggatgtgaactgagagtgaagttcagcaggag cgcagacgcccccgcgtaccagcagggccagaaccagctctataacgagctcaatctagg acgaagagaggagtacgatgttttggacaagagacgtggccgggaccctgagatgggggg aaagccgagaaggaagaaccctcaggaaggcctgtacaatgaactgcagaaagataag atggcggaggcctacagtgagattgggatgaaaggcgagcgccggaggggcaaggggc acgatggcctttaccagggtctcagtacagccaccaaggacacctacgacgcccttcacatg caggccctgccccctcgc |
| SEQ ID NO.21 | gatattgtgctgacccagagccccaagttcatgagcaccagcgtgggcgatagagtgagcgt gacctgcaaggcaagccagaacgtgggaacaaacgtggcctggtaccaacagaaacccg gccaaagccctaagcccctgatttacagcgccacctacagaaatagcggcgtgcccgacag atttacaggaagcggcagcggaaccgatttcacactgaccatcaccaacgtgcagagcaaa gacctggccgactacttctgccagcagtacaacagatacccctacaccagcggaggagga acaaagctggagatcaagagaggtggtggtggttctggcggcggcggctccggtggtggtg gttctcaagtgcaactgcaacagagcggagccgaactggtgagacccggaagcagcgtga agatcagctgcaaggcttccggctacgcctttagcagctactggatgaactgggtgaagcag |
| | agacctggacagggactggaatggatcggccagatttaccctggagacggcgacacaaac tacaacggcaagttcaagggccaagctacactgaccgccgacaaaagcagcagcaccgc ctatatgcagctgagcggactgaccagcgaagatagcgctgtgtacttctgcgccagaaaga ccatcagcagcgtggtggacttctacttcgactactggggacaaggcaccaccctgacagtg agcagcttcgaaaccacgacgccagcgccgcgaccaccaacaccggcgcccaccatcgc gtcgcagcccctgtccctgcgcccagaggcgtgccggccagcggcggggggcgcagtgca cacgagggggctggacttcgcctgtgatatctacatctgggcgcccttggccgggacttgtggg gtccttctcctgtcactggttatcaccctttactgcaaacggggcagaaagaaactcctgtatata ttcaaacaaccatttatgagaccagtacaaactactcaagaggaagatggctgtagctgccg atttccagaagaagaagaaggaggatgtgaactgagagtgaagttcagcaggagcgcaga cgcccccgcgtaccagcagggccagaaccagctctataacgagctcaatctaggacgaag agaggagtacgatgttttggacaagagacgtggccgggaccctgagatggggggaaagcc gagaaggaagaaccctcaggaaggcctgtacaatgaactgcagaaagataagatggcgg aggcctacagtgagattgggatgaaaggcgagcgccggaggggcaaggggcacgatgg cctttaccagggtctcagtacagccaccaaggacacctacgacgcccttcacatgcaggccc tgccccctcgcgcggccgctgagggcagaggaagtcttctaacatgcggtgacgtggagga gaatcccggcccttccggagccaccatggccttaccagtgaccgccttgctcctgccgctggc cttgctgctccacgccgccaggccgggatccgacattgagctcacccagtctccatcctcccta gctgtgtcagttggagagaaggttactatgagctgcaagtccagtcagagccttttatatagtag caatcaaaagaactatttggcctggtaccagcagaaaccagggcagtctcctaaactgctga tttactgggcatccactagggaatctggggtccctgatcgcttcacaggcagtggatctgggac agatttcactctcaccgtcagcagtgtgaaggctgaagacctggcagtttattactgtcagcaat cttatagttatccattcacattcggctcgggcaccaagctggaaatcaaacggggtggtggtgg ttctggcggcggcggctccggtggtggtggttctcaggtcaaactgcagcagtcaggacctga actggtgaagcctggggcctcagtgaagatttcctgcaaagcttctggctacgatttcagtatttc ttggatgaactgggtgaggcagaggcctggacagggtcttgagtggattggacggatttatcct ggagatggagatagtaactacaatgggaagttcgagggcaaggccacactgactgcagac aaatcctccagcacagcctacatgcagctcagcggcctgacctctgtggactctgcggtctattt ttgtgcaagaaccaccactatgattgccctctatgctatggactactggggccaagggaccac ggtcaccgtctcctcagaattcaccacgacgccagcgccgcgaccaccaacaccggcgcc caccatcgcgtcgcagcccctgtccctgcgcccagaggcgtgccggccagcggcgggggg cgcagtgcacacgagggggctggacttcgcctgtgatatctacatctgggcgcccttggccgg gacttgtggggtccttctcctgtcactggttatcaccctttactgcaggagtaagaggagcaggc |
| | tcctgcacagtgactacatgaacatgactccccgccgccccgggcccacccgcaagcattac cagccctatgccccaccacgcgacttcgcagcctatcgctccagagtgaagttcagcaggag cgcagacgcccccgcgtaccagcagggccagaaccagctctataacgagctcaatctagg acgaagagaggagtacgatgttttggacaagagacgtggccgggaccctgagatgggggg aaagccgagaaggaagaaccctcaggaaggcctgtacaatgaactgcagaaagataag atggcggaggcctacagtgagattgggatgaaaggcgagcgccggaggggcaaggggc acgatggcctttaccagggtctcagtacagccaccaaggacacctacgacgcccttcacatg caggccctgccccctcgc |
| SEQ ID NO.22 | gatattgtgctgacccagagccccaagttcatgagcaccagcgtgggcgatagagtga gcgtgacctgcaaggcaagccagaacgtgggaacaaacgtggcctggtaccaacagaaa cccggccaaagccctaagcccctgatttacagcgccacctacagaaatagcggcgtgcccg acagatttacaggaagcggcagcggaaccgatttcacactgaccatcaccaacgtgcagag caaagacctggccgactacttctgccagcagtacaacagatacccctacaccagcggagg aggaacaaagctggagatcaagagaggtggtggtggttctggcggcggcggctccggtggt ggtggttctcaagtgcaactgcaacagagcggagccgaactggtgagacccggaagcagc gtgaagatcagctgcaaggcttccggctacgcctttagcagctactggatgaactgggtgaag cagagacctggacagggactggaatggatcggccagatttaccctggagacggcgacaca aactacaacggcaagttcaagggccaagctacactgaccgccgacaaaagcagcagcac cgcctatatgcagctgagcggactgaccagcgaagatagcgctgtgtacttctgcgccagaa agaccatcagcagcgtggtggacttctacttcgactactggggacaaggcaccaccctgaca gtgagcagcttcgaaaccacgacgccagcgccgcgaccaccaacaccggcgcccaccat cgcgtcgcagcccctgtccctgcgcccagaggcgtgccggccagcggcggggggcgcagt gcacacgagggggctggacttcgcctgtgatatctacatctgggcgcccttggccgggacttgt ggggtccttctcctgtcactggttatcaccctttactgcaggagtaagaggagcaggctcctgca cagtgactacatgaacatgactccccgccgccccgggcccacccgcaagcattaccagccc tatgccccaccacgcgacttcgcagcctatcgctccagagtgaagttcagcaggagcgcag acgcccccgcgtaccagcagggccagaaccagctctataacgagctcaatctaggacgaa gagaggagtacgatgttttggacaagagacgtggccgggaccctgagatggggggaaagc cgagaaggaagaaccctcaggaaggcctgtacaatgaactgcagaaagataagatggcg gaggcctacagtgagattgggatgaaaggcgagcgccggaggggcaaggggcacgatg gcctttaccagggtctcagtacagccaccaaggacacctacgacgcccttcacatgcaggcc ctgccccctcgcgcggccgctgagggcagaggaagtcttctaacatgcggtgacgtggagg |
| | agaatcccggcccttccggagccaccatggccttaccagtgaccgccttgctcctgccgctgg ccttgctgctccacgccgccaggccgggatccgacattgagctcacccagtctccatcctccct agctgtgtcagttggagagaaggttactatgagctgcaagtccagtcagagccttttatatagta gcaatcaaaagaactatttggcctggtaccagcagaaaccagggcagtctcctaaactgctg atttactgggcatccactagggaatctggggtccctgatcgcttcacaggcagtggatctggga cagatttcactctcaccgtcagcagtgtgaaggctgaagacctggcagtttattactgtcagca atcttatagttatccattcacattcggctcgggcaccaagctggaaatcaaacggggtggtggt ggttctggcggcggcggctccggtggtggtggttctcaggtcaaactgcagcagtcaggacct gaactggtgaagcctggggcctcagtgaagatttcctgcaaagcttctggctacgatttcagtat ttcttggatgaactgggtgaggcagaggcctggacagggtcttgagtggattggacggatttat cctggagatggagatagtaactacaatgggaagttcgagggcaaggccacactgactgcag acaaatcctccagcacagcctacatgcagctcagcggcctgacctctgtggactctgcggtct atttttgtgcaagaaccaccactatgattgccctctatgctatggactactggggccaagggacc acggtcaccgtctcctcagaattcaccacgacgccagcgccgcgaccaccaacaccggcg cccaccatcgcgtcgcagcccctgtccctgcgcccagaggcgtgccggccagcggcgggg ggcgcagtgcacacgagggggctggacttcgcctgtgatatctacatctgggcgcccttggcc gggacttgtggggtccttctcctgtcactggttatcaccctttactgcaggagtaagaggagcag gctcctgcacagtgactacatgaacatgactccccgccgccccgggcccacccgcaagcatt accagccctatgccccaccacgcgacttcgcagcctatcgctccagagtgaagttcagcagg agcgcagacgcccccgcgtaccagcagggccagaaccagctctataacgagctcaatcta ggacgaagagaggagtacgatgttttggacaagagacgtggccgggaccctgagatgggg ggaaagccgagaaggaagaaccctcaggaaggcctgtacaatgaactgcagaaagata agatggcggaggcctacagtgagattgggatgaaaggcgagcgccggaggggcaaggg gcacgatggcctttaccagggtctcagtacagccaccaaggacacctacgacgcccttcaca tgcaggccctgccccctcgc |
| SEQ ID NO.23 | DIVLTQSPKFMSTSVGDRVSVTCKASQNVGTNVAWYQQKPGQSP KPLIYSATYRNSGVPDRFTGSGSGTDFTLTITNVQSKDLADYFCQQ YNRYPYTSGGGTKLEIKRGGGGSGGGGSGGGGSQVQLQQSGAE LVRPGSSVKISCKASGYAFSSYWMNWVKQRPGQGLEWIGQIYPG DGDTNYNGKFKGQATLTADKSSSTAYMQLSGLTSEDSAVYFCAR KTISSVVDFYFDYWGQGTTLTVSS |
| SEQ ID NO.24 | DIELTQSPSSLAVSVGEKVTMSCKSSQSLLYSSNQKNYLAWY QQKPGQSPKLLIYWASTRESGVPDRFTGSGSGTDFTLTVSSVKAE DLAVYYCQQSYSYPFTFGSGTKLEIKRGGGGSGGGGSGGGGSQ VKLQQSGPELVKPGASVKISCKASGYDFSISWMNWVRQRPGQGL EWIGRIYPGDGDSNYNGKFEGKATLTADKSSSTAYMQLSGLTSVD SAVYFCARTTTMIALYAMDYWGQGTTVTVSS |
| SEQ ID NO.25 | TTTPAPRPPTPAPTIASQPLSLRPEACRPAAGGAVHTRGLDFACDI YIWAPLAGTCGVLLLSLVITLYCKRGRKKLLYIFKQPFMRPVQTTQE EDGCSCRFPEEEEGGCELRVKFSRSADAPAYQQGQNQLYNELNL GRREEYDVLDKRRGRDPEMGGKPRRKNPQEGLYNELQKDKMAE AYSEIGMKGERRRGKGHDGLYQGLSTATKDTYDALHMQALPPR |
| SEQ ID NO.26 | EGRGSLLTCGDVEENPGP |

### DETAILED DESCRIPTION OF THE INVENTION

The present invention discloses a CAR targeting CD22 and CD19 and application thereof, and those skilled in the art can learn from the content herein to appropriately modify the process parameters to achieve the same. It should be specifically noted that all such alternatives and modifications as would be obvious to those skilled in the art are deemed to be included in the present invention and that it is obvious to those skilled in the relevant art can implement and apply the technology of the present invention by making modifications or appropriate changes and combinations on the content described herein without departing from the content, spirit and scope of the present invention.

In the present invention, unless otherwise specified, scientific and technical terms used herein have the meanings commonly understood by those skilled in the art.

In order to make those skilled in the art better understand the technical solution of the present invention, the present invention will be further described in detail below with reference to specific embodiments.

### Example 1: Construction of a Dual-Target Chimeric Antigen Receptor Vector

1. BamH I and EcoR I endonucleases were adopted to digest a plasmid containing CD8α-4-1BB-CD3ζ segment constructed by the inventor previously to obtain a CD8a-4-1BB-CD3ζ segment, an amino acid sequence of which is shown in SEQ ID NO.5, where the plasmid containing the CD8α-4-1BB-CD3ζ segment may be prepared by any suitable method in the prior art, for example, the patent No. ZL201510233748.0.

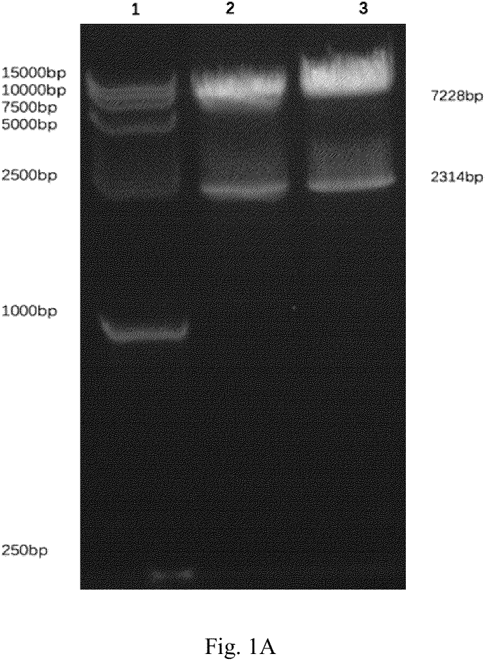

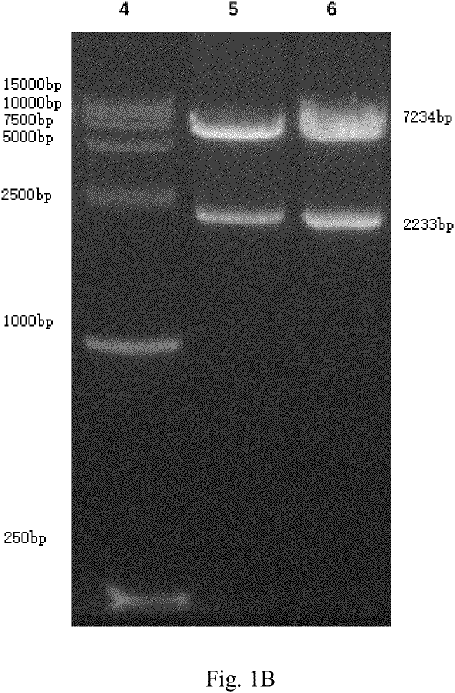

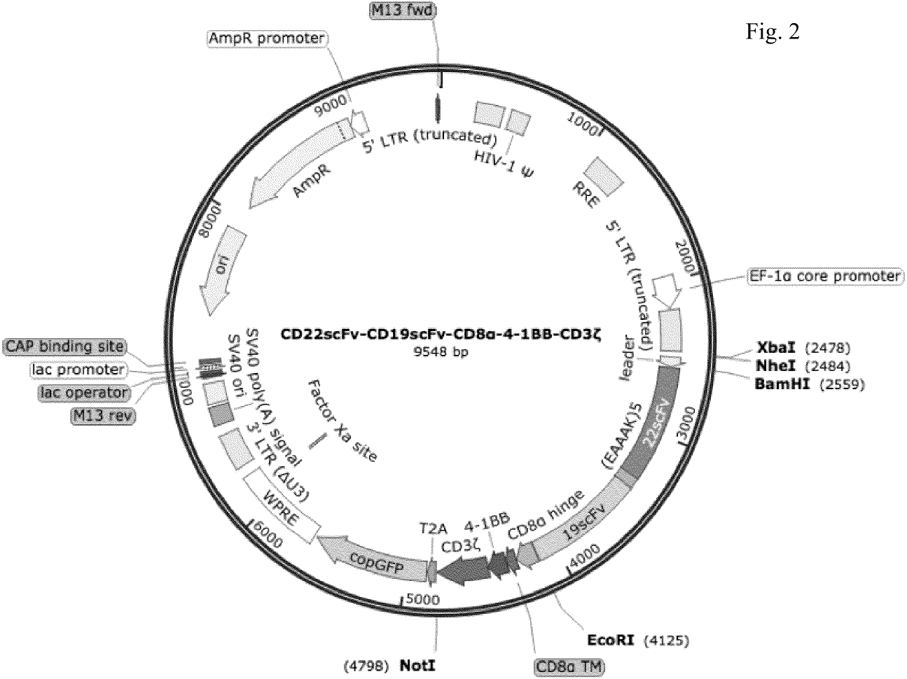

2. CD22scFv-CD19scFv, CD19scFv-CD22scFv, CD19V_{L}-CD22V_{L}-CD22V_{H}-CD19V_{H} and CD22V_{L}-CD19V_{L}-CD19V_{H}-CD22V_{H} segments obtained by synthesis were incorporated to a target vector, respectively, and constructed CD22scFv-CD19 scFv-CD8α-4-1BB-CD3ζ CAR (22-19 CAR), CD19scFv-CD22 scFv-CD8α-4-1BB-CD3ζ CAR (19-22 CAR), CD19 V_{L}-CD22V_{L}-CD22V_{H}-CD19V_{H}-CD8α-4-1BB -CD3ζ (19×22 CAR) and CD22 V_{L}-CD19 V_{L}-CD19V_{H}-CD22V_{H}-CD8α-4-1BB-CD3ζ (22×19 CAR) target vectors were digested with endonucleases Nhe I and Not I for identification. The results are shown in FIG. 1A and FIG. 1B. The results of digestion showed that positive clones contained a target band and were correct after sequencing and identification. A schematic diagram of a vector of CD22 scFv-CD19 scFv-CD8α-4-1 BB-CD3ζ CAR is taken as an example, as shown in FIG. 2.

### Example 2: Preparation of a Lentivirus-Modified T Cells of a Dual-Target Chimeric Antigen Receptor

1. An EndoFree Plasmid Maxi plasmid extraction kit (QIAGEN) was used to respectively extract 22-19 CAR, 19-22-CAR, 19×22 CAR and 22×19 CAR expression plasmids and packaging plasmids PRSV-Rev, pMDlg-PRRE and pMD.2G. Each CAR plasmid and packaging plasmid (four plasmids) were transfected with a PEI transfection reagent (Polyscience) in a ratio of 12.2: 4.11: 8.75: 3.5 (refer to the instructions of the PEI transfection reagent for specific methods). A fresh culture medium was replaced 12 hours after transfection, virus supernatant was collected in 24 hours and 48 hours, respectively, centrifuged at 4° C. by 3000 rpm for 15 minutes, and filtered through a 0.45 µm filter, and then virus supernatant was ultracentrifuged at 4° C. by 50000 g for 1.5 hours to concentrate for 10 folds, and then stored at -80° C.

2. Preparation of the T cells: 10 ml of fresh healthy human peripheral blood was obtained, and T cells were purified using RosetteSep T cell enrichment Cocktail (Stemcell) and Ficoll-Paque PLUS (GE Healthcare) (for specific steps, follow the instructions of RosetteSep T cell enrichment Cocktail). Anti-CD3/CD28 magnetic beads (Gibco) were added according to the ratio of cells: magnetic beads=1:1, and cultured for 24 hours to obtain T cells before transfection.

3. Lentivirus-infected T cells and culture of post-infected T cells: viral supernatant was taken out from -80° C. freezer, thawed at room temperature, 1×10⁶ T cells were transfected with 100 µl of viral supernatant, and Polybrene was added till a final concentration reached 8 µg/ ml. The transfected T cells were centrifuged at 32° C. at a speed of 1800 rpm for 1.5 hours, and then cultured in an incubator with 5% CO₂ at 37° C.

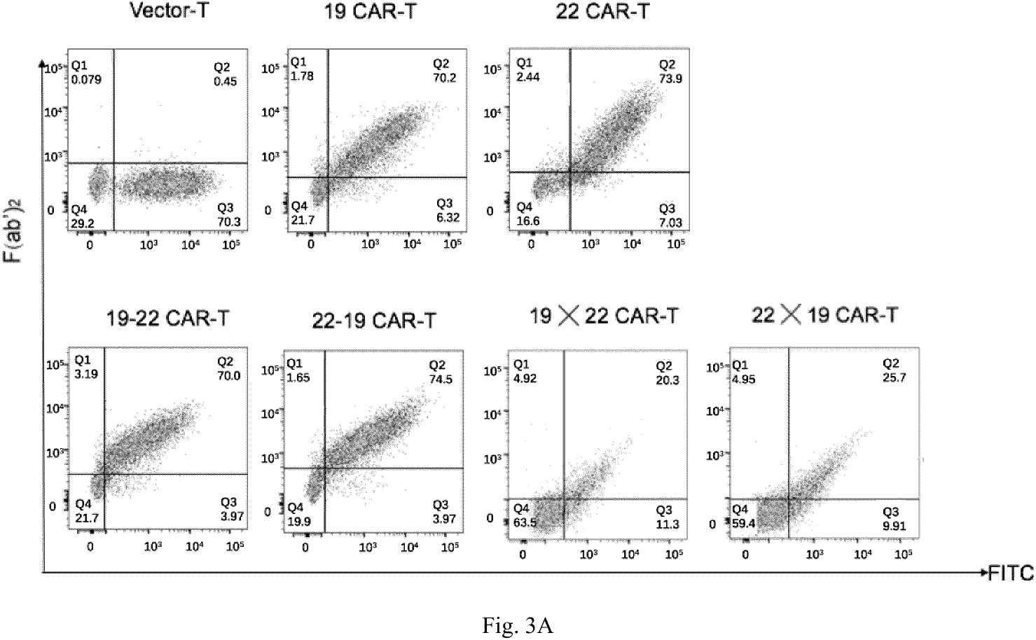

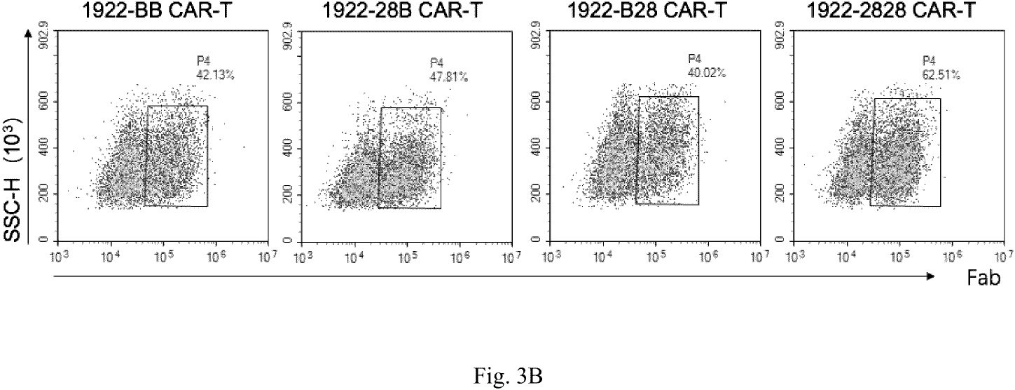

4. Detection of the positive rate of CAR-modified T cells by flow cytometry: the above culture T cells were collected and labeled with a rabbit anti-mouse IgG F(ab′)₂ antibody, the F(ab′)₂ and GFP positive T cells were analyzed by flow cytometry. The results are shown in FIG. 3A and FIG. 3B. It is observed from the figures that the positive rate of CAR-T is 70% or above.

### Experimental Example 1: The Killing Effect of 22-19 CAR, 19-22-CAR, 19×22 CAR and 22×19 CAR Lentivirus-Modified T Cells on Leukemia Cells

### 1. Expression Levels of CD22 in Hematological Tumor Cell Lines

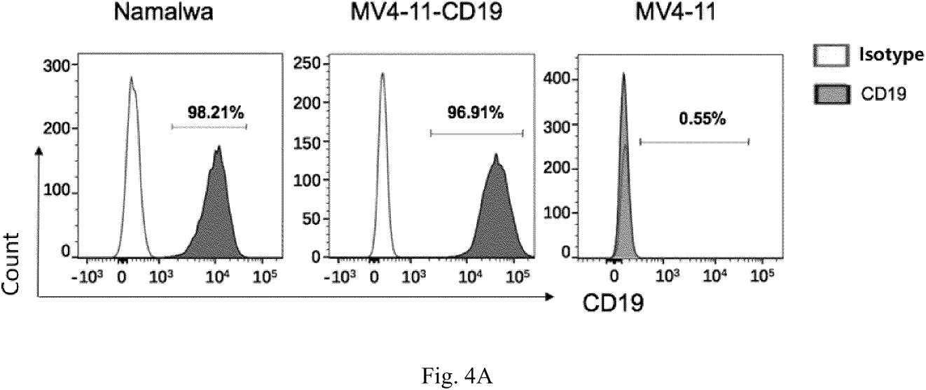

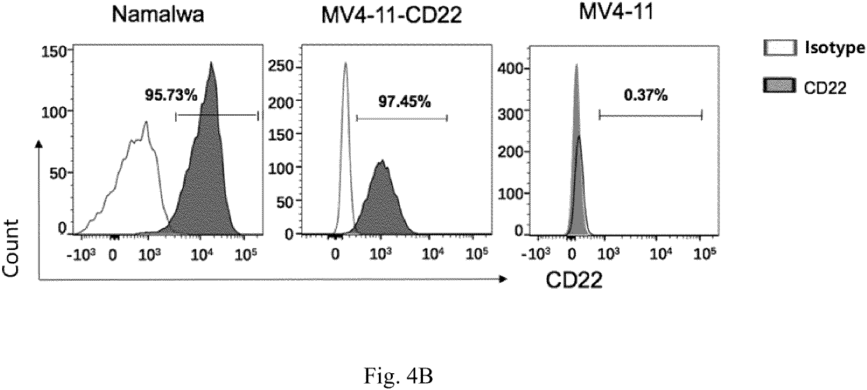

Namalwa and MV4-11 cell lines were purchased from ATCC in the United States. MV4-11-CD19 and MV4-11-CD22 were monoclonal cell lines screened after the MV4-11 cell lines were infected with CD19 and CD22, respectively. 5×10⁵ cells were collected from each of the cultured cell lines, and washed twice with PBS, a PE conjugated anti-human CD19 monoclonal antibody and an APC conjugated anti-human CD22 monoclonal antibody (Biolegend) were stained respectively, PE-isotype and APC-isotype were used as control groups, and then incubated on ice for 30 minutes. The expression levels of CD19 and CD22 in various cell lines were detected by flow cytometry, and the results are shown in FIG. 4A and FIG. 4B, where FIG. 4A was the positive rate of CD19 antigen expression; FIG. 4B was the positive rate of CD22 antigen expression; and the CD19 positive rates of Namalwa and MV4-11-CD19 were both 95% or above, the CD22 positive rates of Namalwa and MV4-11-CD22 were both 95% or above, while wild-type MV4-11 hardly expressed CD19 and CD22.

### 2. Detection of Residual Tumor Cells by Flow Cytometry After Co-Culture of CAR-Modified T Cells With Namalwa, MV4-11-CD19, MV4-11-CD22 and Wild-Type MV4-11 Cell Lines

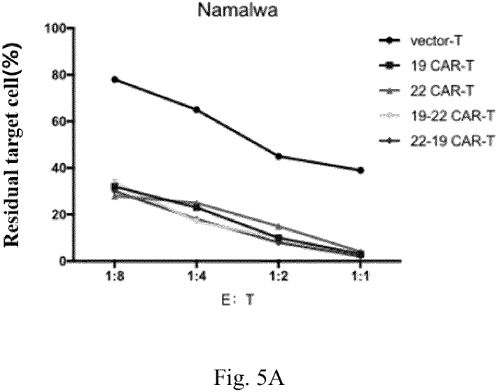

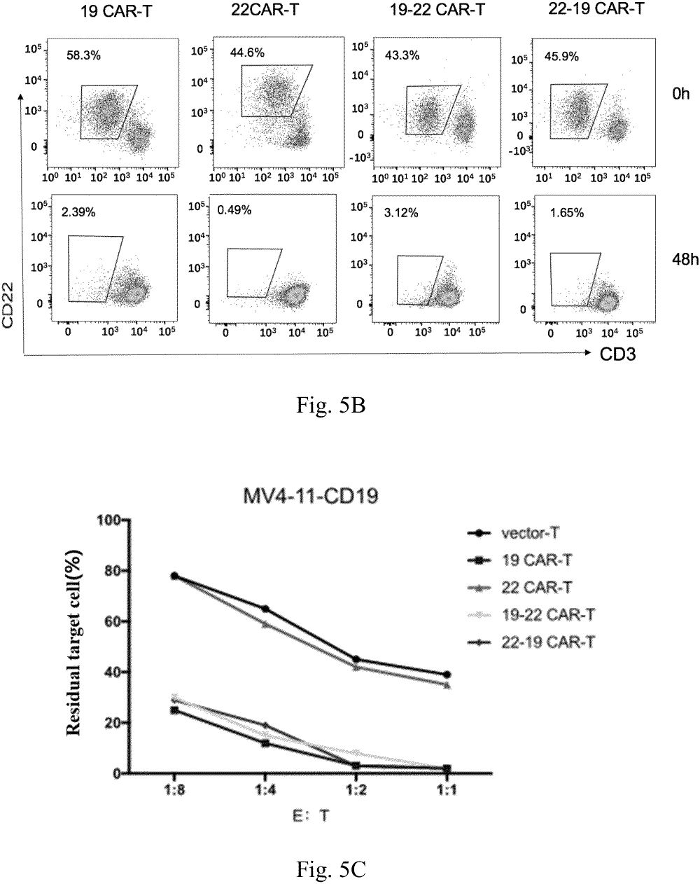

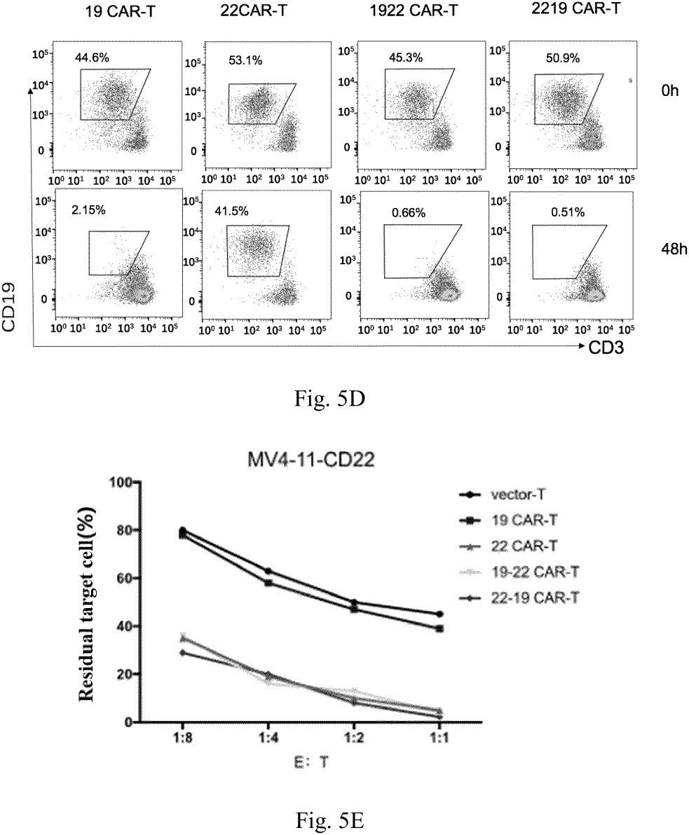

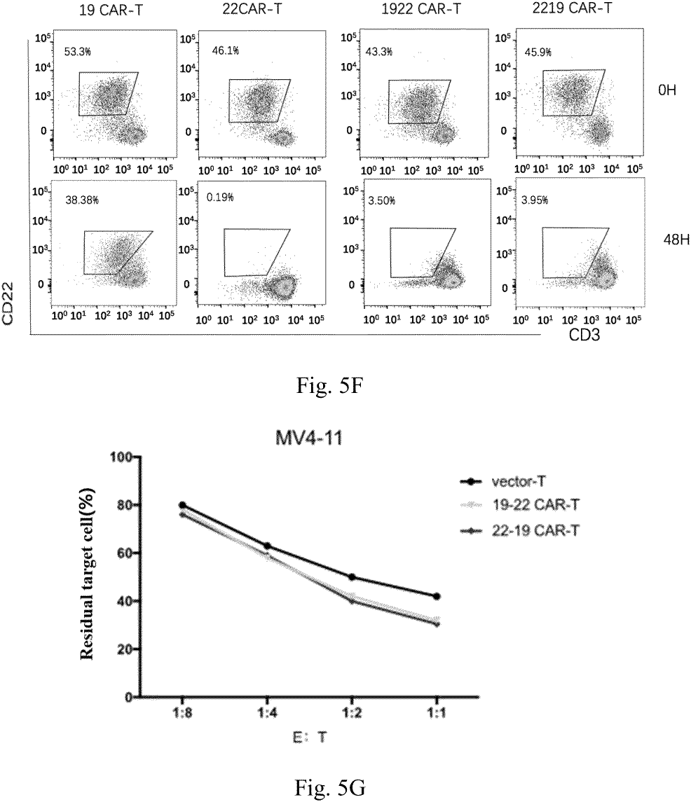

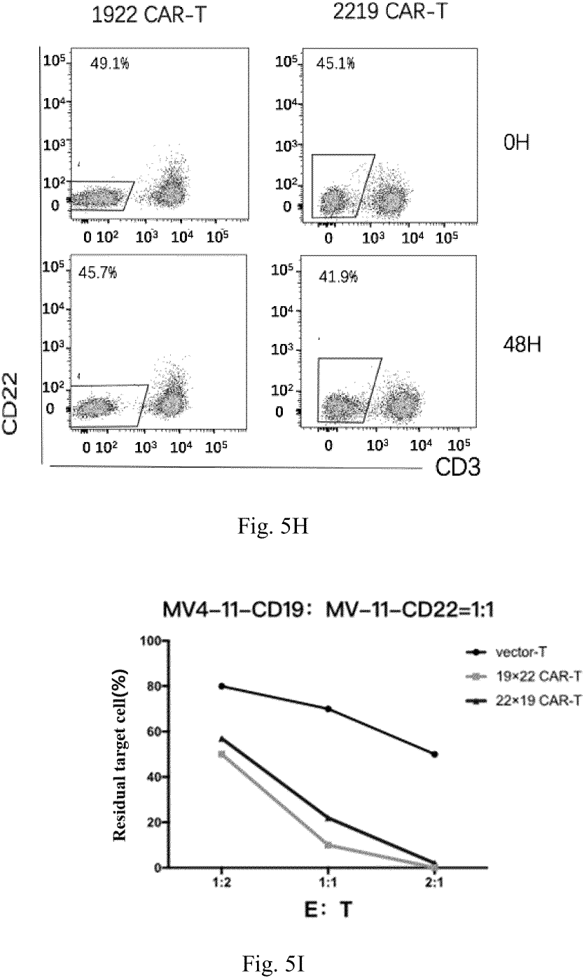

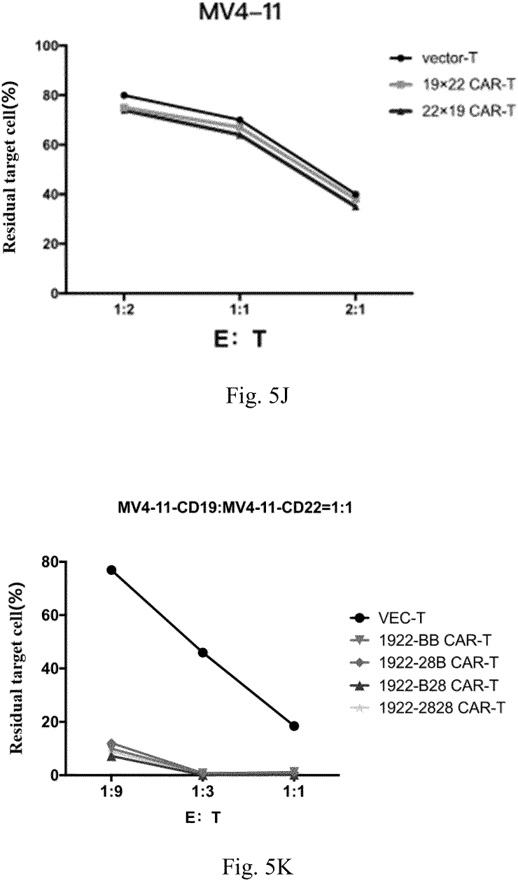

The above cells were inoculated into a 24-well culture plate at 2×10⁵ cells per well, and CAR-T cells of 2.5×10⁴ (E:T=1:8), 5×10⁴ (E:T=1:4), and 1×10⁵ (E: T=1:2), 2×10⁵ (E:T=1:1), and 4×10⁵ (E:T=2:1) were added, respectively, empty vector-T cells were used as a control group, and co-cultured in an incubator. The co-cultured Namalwa, MV4-11-CD19, MV4-11-CD22 and wild-type MV4-11 cells were stained with PE conjugated anti-human CD19 monoclonal antibody and the APC conjugated anti-human CD22 monoclonal antibody (Biolegend), and PE-Cy7 anti-human CD3 monoclonal antibody (Biolegend) were used to label the T cells, and the residual cells were tested by flow cytometry. The results are shown in FIG. 5A to FIG. 5L. 1) Compared with vector-T, 19 CAR-T, 22 CAR-T, 19-22 CAR-T and 22-19 CAR-T could significantly kill CD19⁺CD22⁺ Namalwa target cells (FIG. 5A and FIG. 5B). 2) Compared with vector-T and 22 CAR-T, 19 CAR-T, 19-22 CAR-T and 22-19 CAR-T could significantly kill MV4-11-CD19 target cells (FIG. 5C and FIG. 5D). 3) Compared with vector-T and 19 CAR-T, 22 CAR-T, 19-22 CAR-T and 22-19 CAR-T could significantly kill MV4-11-CD22 target cells (FIG. 5E and FIG. 5F). 4) Similar to vector-T, 19-22 CAR-T and 22-19 CAR-T had no killing effect on CD19/CD22 double negative MV4-11 target cells (FIG. 5G and FIG. 5H). 5) Compared with vector-T, both 19×22 CAR-T and 22×19 CAR-T could significantly kill the MV4-11-CD19 target cells and the MV4-11-CD22 target cells (FIG. 5I). 6) Similar to vector-T, 19×22 CAR-T and 22×19 CAR-T had no killing effect on the CD19/CD22 double-negative MV4-11 target cells (FIG. 5J). 7) Compared with vector-T, 1922-BB CAR-T, 1922-28B CAR-T, 1922-B28 CAR-T and 1922-2828 CAR-T could significantly kill the MV4-11-CD19 target cells and the MV4-11-CD22 target cells (FIG. 5K). 8) Similar to vector-T, 1922-BB CAR-T, 1922-28B CAR-T, 1922-B28 CAR-T and 1922-2828 CAR-T had no killing effect on the CD19/CD22 double negative MV4-11 target cells (FIG. 5L). The results proved that 19-22 CAR-T, 22-19 CAR-T, 19×22 CAR-T, 22×19 CAR-T, 1922-BB CAR-T, 1922-28B CAR-T, 1922-B28 CAR-T and 1922-2828 CAR-T had obvious dual-target advantages, and had efficient and specific killing effects on CD19⁺, CD22⁺ and CD19⁺/CD22⁺ target cells. In the above experiments, the killing effects of 22-19 CAR-T and 22×19 CAR-T were more significant.

### 3. Degranulation Assays to Analyze the Activation of CAR-Modified T Cells

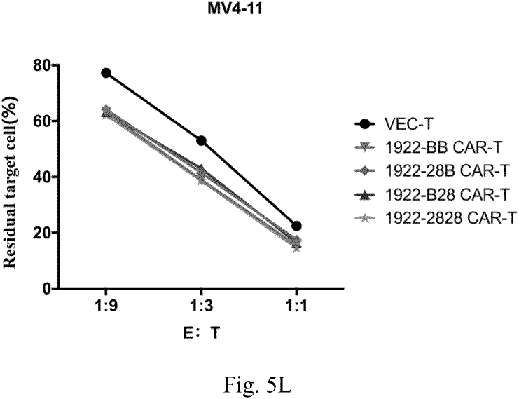

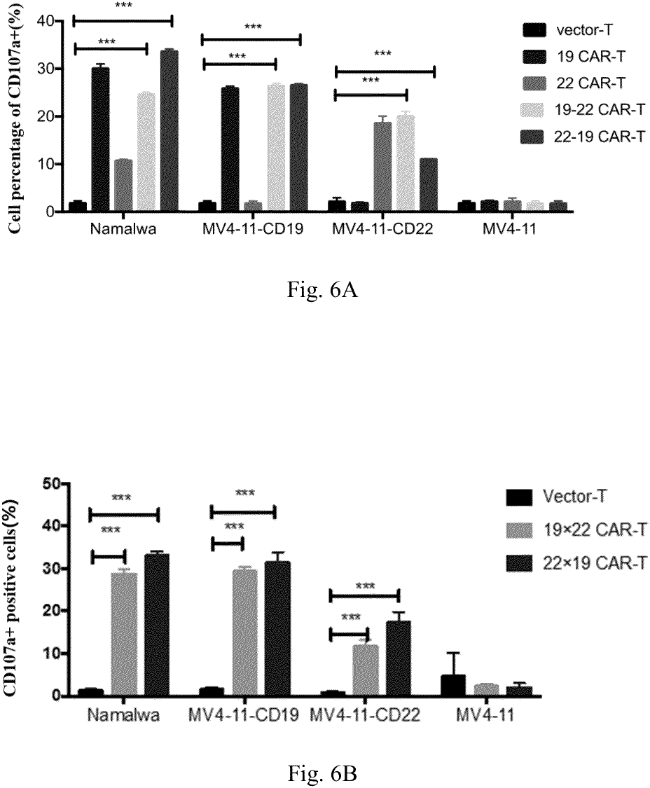

CAR-T and vector-T cells were co-cultured with Namalwa, MV4-11-CD19, MV4-11-CD22 and wild-type MV4-11 cell lines respectively according to the E:T ratio of 1:1, and an anti-CD107a antibody and monensin were added to the co-culture system; and the expression level of CD107a on the surface of GFP⁺ cells was tested by flow cytometry after 4h. The results are shown in FIG. 6A and FIG. 6B. 1) After co-culture with Namalwa cells (CD19⁺CD22⁺), the degranulation levels of 19 CAR-T, 22 CAR-T, 19-22 CAR-T, 22-19 CAR-T, 19×22 CAR-T and 22×19 CAR-T groups were all 10% or above, and the degranulation level of vector-T was 2% or below. 2) After co-culture with MV4-11-CD19 cells, the degranulation levels of 19 CAR-T, 19-22 CAR-T, 22-19 CAR-T, 19×22 CAR-T and 22×19 CAR-T groups were all 25% or above, while the degranulation levels of vector-T and 22 CAR-T were 2% or below; 3) after co-culture with target cells MV4-11-CD22, the degranulation levels of 22 CAR-T, 19-22 CAR-T, 22-19 CAR-T, 19×22 CAR-T and 22×19 CAR-T groups were all 15% or above, while the degranulation levels of vector-T and 19 CAR-T were 2% or below. 4) After co-culture with wild-type MV4-11, the degranulation levels of vector-T, 19 CAR-T, 22 CAR-T, 19-22 CAR-T, 19×22 CAR-T and 22×19 CAR-T were all 2% or below, demonstrating that 19-22 CAR-T, 22-19 CAR-T, 19×22 CAR-T and 22×19 CAR-T could be significantly and specifically activated by CD19⁺ or CD22⁺ target cells.

### 4. ELISA to Detect the Levels of Cytokines IFN-γ, TNF-α and IL-2 in Co-Culture Supernatant of Lymphoma Cell Lines and CAR-T Cells

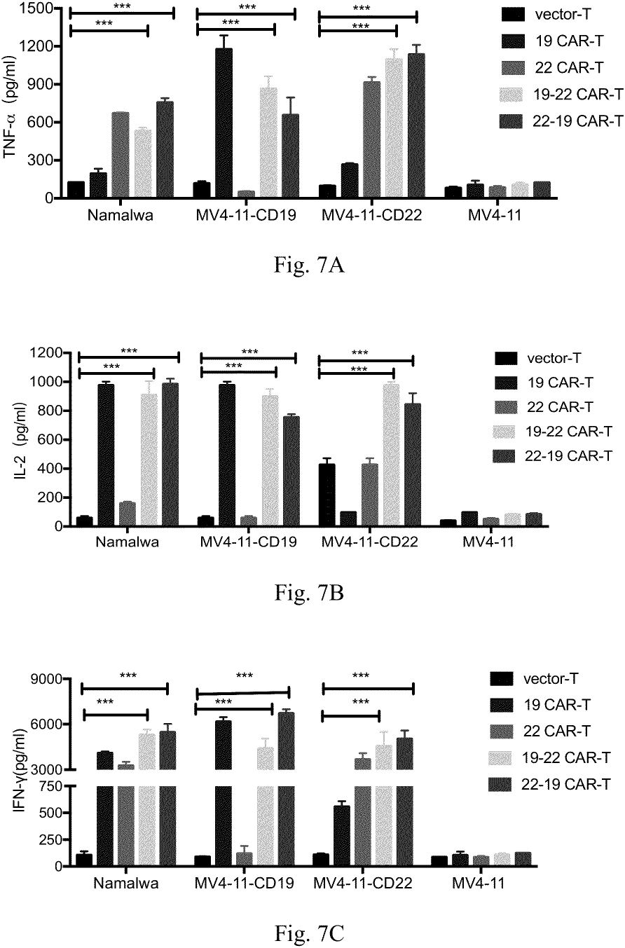

Namalwa, MV4-11-CD19, MV4-11-CD22 and wild-type MV4-11 cell lines were inoculated in 24-well plates at 2×10⁵ cells per well, and CAR-T and vector-T cells were added at 2×10⁵ cells to each well, the culture medium was supplemented to 1 ml, and co-cultured in an incubator for 24 hours. Human IFN-γ, TNF-α and IL-2 ELISA assay kits (R&D) were used to test the cytokine secretion level in the co-culture supernatant (see instructions of the ELISA assay kits for specific steps). The results are shown in FIG. 7A to FIG. 7C. 1) After co-culture with CD19⁺CD22⁺ Namalwa cells, the release levels of all three cytokines were significantly higher in 19 CAR-T, 19-22 CAR-T and 22-19 CAR-T groups compared with that of vector-T cells, the secretion levels of TNF-α were significantly higher in the 19-22 CAR-T and 22-19 CAR-T groups than in the 19 CAR-T group, and the secretion levels of IL-2 were significantly higher in the 19-22 CAR-T and 22-19 CAR-T groups than in a 22 CAR-T group. 2) After co-culture with MV4-11-CD19, the release levels of the three cytokines were significantly higher in the 19 CAR-T, 19-22 CAR-T, and 22-19 CAR-T groups than in the vector-T and 22 CAR-T groups. 3) After co-culture with MV4-11-CD22, the release levels of TNF-α and IFN-_{Y} were significantly higher in the 22 CAR-T, 19-22 CAR-T, and 22-19 CAR-T groups than in the vector-T and 19 CAR-T groups; while it could be seen that the secretion levels of IL-2 were higher in the 19-22 CAR-T and 22-19 CAR-T groups than in the 22 CAR-T group. 4) After co-culture with wild-type MV4-11, the release levels of all three cytokines were low in vector-T, 19 CAR-T, 22 CAR-T, and 19-22 CAR-T. It demonstrated that 19-22 CAR-T and 22-19 CAR-T could specifically activate CD19⁺, CD22⁺ and CD19⁺CD22⁺ target cells and release cytokines, and exhibited higher release levels of cytokines than 19 CAR-T or 22 CAR-T.

### 5. Expression Levels of CD19 and CD22 in Bone Marrow Mononuclear Cells (BMMCs) of ALL Patients

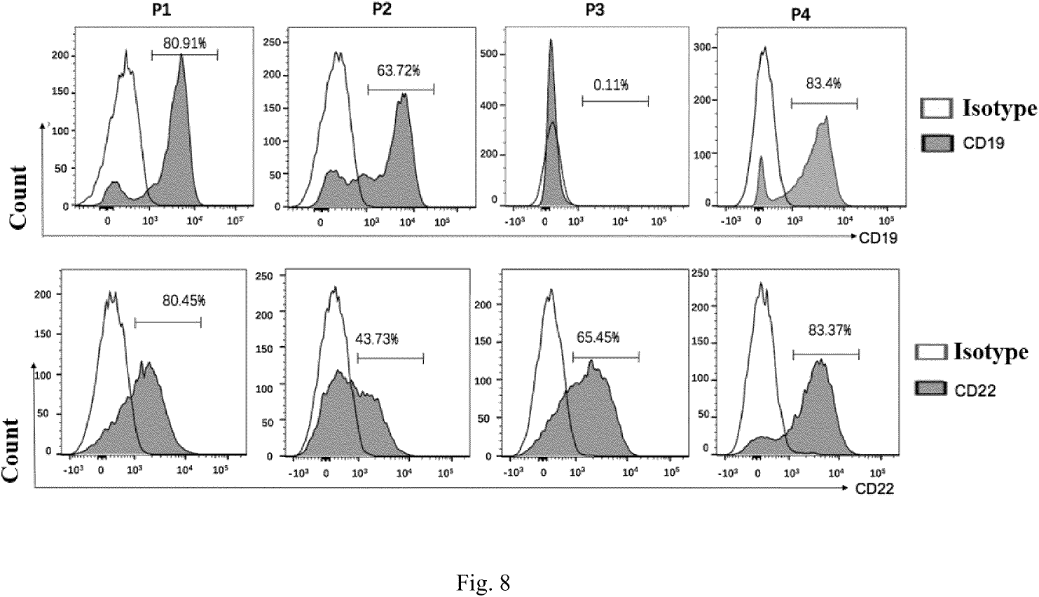

Patient samples were obtained from the Institute of Hematology and Blood Diseases Hospital, CAMS, and informed consent was obtained from the patients. The BMMCs were separated by Ficoll gradient centrifugation, 5×10⁵ cells were collected and washed twice with PBS, and then incubated with PE conjugated anti-human CD19 monoclonal antibody, APC conjugated anti-human CD22 monoclonal antibody (Biolegend), PE-isotype and APC-isotype (control groups) on ice for 30 minutes. The proportion and intensity of the BMMCs expressing CD19 and CD22 in each patient were analyzed using flow cytometry, as shown in FIG. 8, where P1-P4 represent patients No. 1-4.

### 6. Detection of Residual Tumor Cells by Flow Cytometry After Co-Culture of CAR-Modified T Cells With BMMCs From ALL Patients

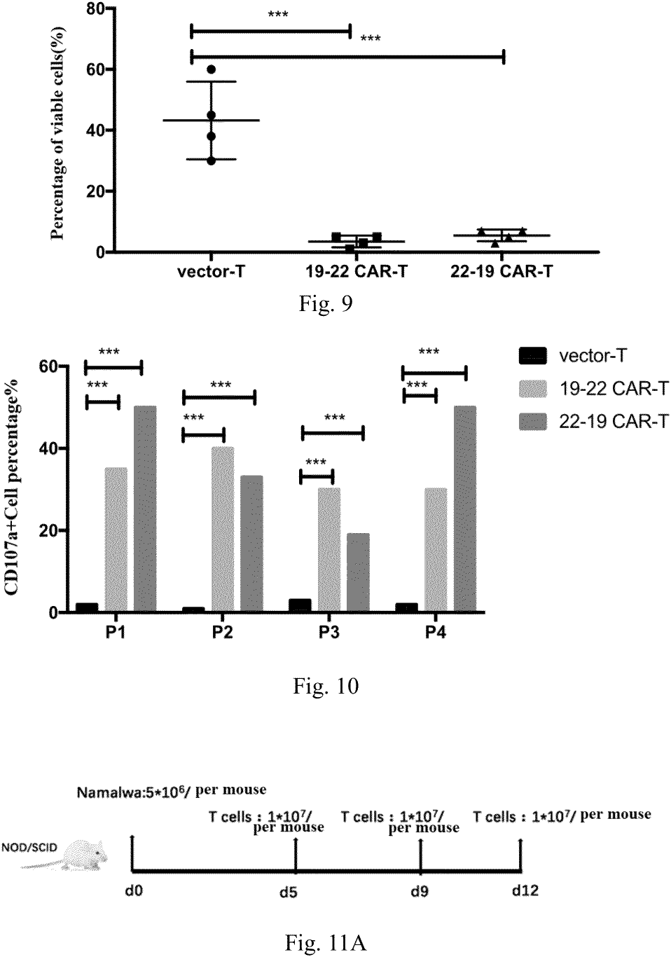

BMMCs were seeded in 24-well culture plates at 4×10⁵ cells/well, 1×10⁵ (E:T=1:4) of CAR-modified T cells were added respectively, empty vector-T cells were used as control group, and then co-cultured in an incubator for 48 h. The co-cultured cells were then stained with PE conjugated anti-human CD19 monoclonal antibody and APC conjugated anti-human CD22 monoclonal antibody (Biolegend) for detection of residual leukemic cells from ALL patients, and APC-Cy7 conjugated anti-human CD3 monoclonal antibody (Biolegend) for detection of T cells, and analyzed by flow cytometry. The results are shown in FIG. 9, which demonstrated that after co-culture of CAR-T with CD19⁺/CD22⁺ BMMCs from patients 1-4 for 48 hours, only 3.03%, 0.24%, 1.34%, and 2.87% of CD19⁺/CD22⁺ cells could survive in 19-22 CAR-T treatment group, 4.10%, 0.45%, 1.68%, and 3.89% of CD19⁺/CD22⁺ cells in 22-19 CAR-T treatment group, respectively, while 46.58%, 63.95%, 23.76%, and 44.88% of CD19⁺/CD22⁺ cells remained in the control group. From above results, it suggested that CAR-T had a killing effect on CD19⁺/CD22⁺ leukemic primary BMMCs.

### 7. Degranulation Assays to Analyze Activation of CAR-Modified T Cells After Co-Cultured With BMMCs From ALL Patients

CAR-T and vector-T cells were co-cultured with BMMCs of patients 1-4 according to the E:T ratio of 1:1, and an anti-CD107a antibody and monensin were added to co-culture systems; and the expression level of CD107a on the surface of CD3⁺ cells was tested by flow cytometry after 4 h. The results are shown in FIG. 10, which revealed that after co-cultured with BMMCs from patients 1-4, the activation percentages of 19-22 CAR-T cells were 35.53%, 40.37%, 31.37% and 30.20%, respectively; and the activation percentages of 22-19 CAR-T cells were 51.21%, 33.04%, 18.01% and 50.23%, respectively; while the activation percentages of vector T-cells were only 3.07%, 1.73%, 3.43%, and 2.40%, respectively. From the above results, it showed that there was a significant difference in the activation of CAR-T and vector-T by cocultured with BMMCs of ALL patients.

### 8. Effects of CAR-Modified T Cells in a CD19⁺/CD22⁺ Lymphoma Mouse Model

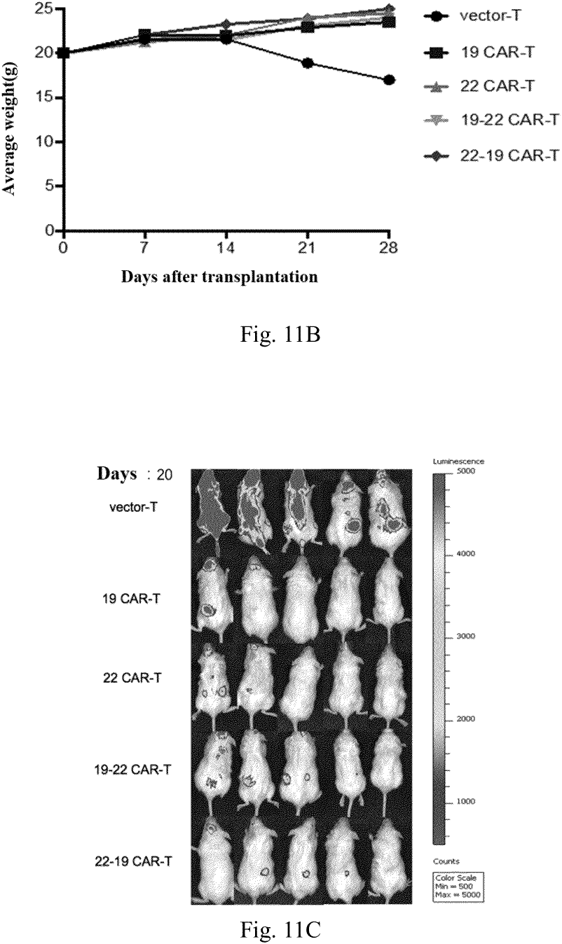

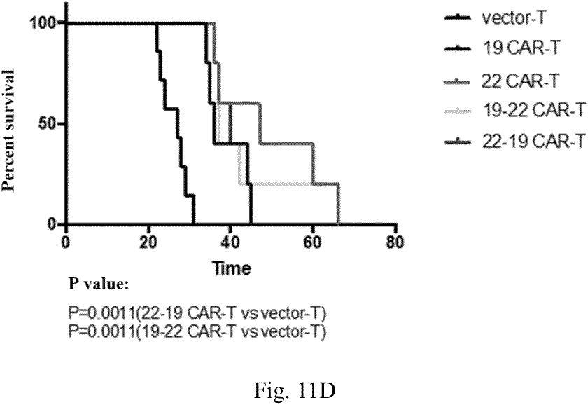

Female NOD/SCID mice at 6-8 weeks were used and randomly divided into vector-T and CAR-T treatment groups, and 5×10⁶ Namalwa cells were inoculated intravenously; and 1×10⁷ vector-T cells or CAR-T cells were intravenously administered on day 5, day 9 and day 12 after transplantation (see FIG. 11A), respectively. Compared with the control group, the mice did not experience significant weight loss due to CAR-T injection, suggesting that CAR-T treatment had no significant toxic side effects on the mice (see FIG. 11B). The median survival times of vector-T group, 19-22 CAR-T group and 22-19 CAR-T group were 25, 36 and 37 days, respectively. The tumor load plots are shown in FIG. 11C, and the tumor loads of the CAR-T groups were significantly lower than that of the vector-T group. The survival curves are shown in FIG. 11D. By calculating the differences of survival times between CAR-T and vector-T treatment groups, it was found that both 19-22 CAR-T and 22-19 CAR-T could significantly prolong the survival times of the mice, and there was a significant statistical difference (p=0.0011) compared with the control group. According to the median survival and tumor load outcome plots, it was found that 22-19 CAR-T had a slightly better treatment effect than that of 19-22 CAR-T.

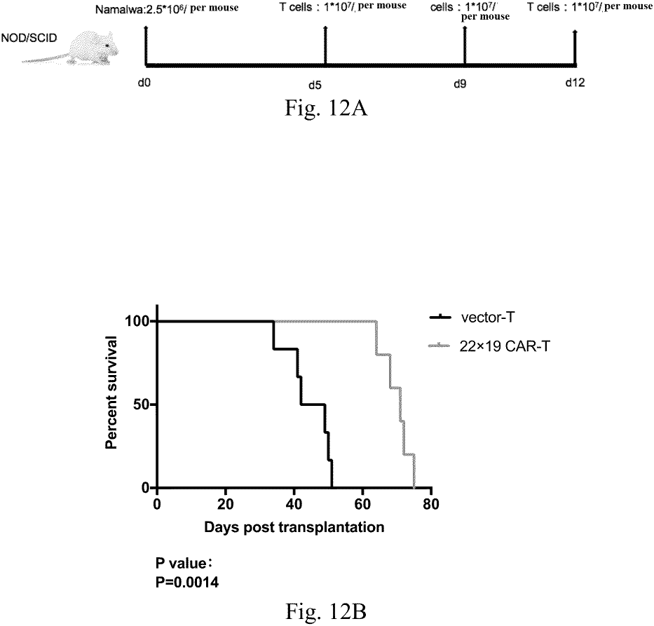

Female NOD/SCID mice at 6-8 weeks were used and randomly divided into two groups, and 2.5×10⁶ Namalwa cells were inoculated intravenously; and 1×10⁷ vector-T cells or CAR-T cells were administered intravenously on day 5, 9 and 12 after transplantation (see FIG. 12A), respectively. The survival curves are shown in FIG. 12B. By calculation the difference of the survival times between the two groups, it was found that 22×19 CAR-T group could significantly prolong the survival time of the mice, and had a significant statistic difference (p=0.0014) compared with that of control group.

The above are only the preferred embodiments of the present invention. It should be noted that those ordinarily skilled in the art can also make various improvements and modifications without departing from the principles of the present invention, and these improvements and modifications should also be regarded as falling within the protection scope of the present invention.

What is claimed is:

1. A nucleic acid molecule for encoding a chimeric antigen receptor targeting CD22 and CD19, wherein the chimeric antigen receptor comprises an extracellular region, a transmembrane region and an intracellular signal transduction region, and the extracellular region encoded by the nucleic acid molecule comprises a CD22 and CD19 binding domain consisting of a CD22 single-chain fragment variable and a CD19 single-chain fragment variable;
and the CD22 single-chain fragment variable and the CD19 single-chain fragment variable are arranged according to an amino acid sequence shown in SEQ ID No.9, an amino acid sequence shown in SEQ ID No.10, an amino acid sequence shown in SEQ ID No.11, an amino acid sequence shown in SEQ ID No. 12, an amino acid sequence shown in SEQ ID No. 23, or an amino acid sequence shown in SEQ ID No. 24 in order.

2. The nucleic acid molecule according to claim 1, wherein the extracellular region encoded by the nucleic acid molecule further comprises a signal peptide constructed at an amino terminal of the chimeric antigen receptor or an amino acid sequence having 90%-99% homology with the signal peptide, and the signal peptide is a signal peptide sequence in CD8α or GM-CSF, preferably signal peptide shown in SEQ ID NO.13.

3. The nucleic acid molecule according to claim 1, wherein the CD22 and CD19 binding domains encoded by the nucleic acid molecule are connected to the transmembrane region encoded by the nucleic acid molecule through a hinge region, and the hinge region is preferably a hinge region sequence in CD8α; and the transmembrane region is a transmembrane domain selected from the following proteins or an amino acid sequence having 90%-99% homology with the proteins: α, β or ζ chains of T cell receptors, CD3ε, CD45, CD4, CD5, CD8, CD9, CD16, CD22, CD33, CD37, CD64, CD80, CD86, CD134, 4-1BB or CD154.

4. The nucleic acid molecule according to claim 1, wherein the intracellular signal transduction region encoded by the nucleic acid molecule further comprises a costimulatory factor.

5. The nucleic acid molecule according to claim 4, wherein the costimulatory factor is one or more of functional signal domains obtained by selecting from the following proteins or the amino acid sequence having 90%-99% homology with the proteins: MHC class I molecules, TNF receptor proteins, immunoglobulin-like proteins, cytokine receptors, integrins, lymphocyte activation signaling molecules, activated NK cell receptors, BTLA, Toll ligand receptors, OX40, CD2, CD7, CD27, CD28, CD30, CD40, CDS, ICAM-1, LFA-1, 4-1BB, B7-H3, CD278, GITR, BAFFR, LIGHT, HVEM, KIRDS2, SLAMF7, NKp80, NKp44, NKp30, NKp46, CD19, CD4, CD8α, CD8β, IL2Rβ, IL2Rγ, IL7Rα, ITGA4, VLA1, CD49α, IA4, CD49D, ITGA6, VLA6, CD49f, ITGAD, CD11d, ITGAE, CD103, ITGAL, CD11α, ITGAM, CD11b, ITGAX, CD11c, CD29, ITGB1, ITGB2, CD18, ITGB7, NKG2D, NKG2C, TNFR2, CD226, CD84, CD96, CEACAM1, CRTAM, CD229, CD160, PSGL1, CD100, CD69, SLAMF6, SLAM, BLAME, CD162, LTBR, LAT, GADS or SLP-76.

6. The nucleic acid molecule according to claim 5, wherein the costimulatory factor is CD28 or 4-1BB or an amino acid sequence having 90%-99% homology therewith.

7. The nucleic acid molecule according to claim 1, wherein a sequence of the nucleic acid molecule is shown in SEQ ID NO.5, SEQ ID NO.6, SEQ ID NO.7 or SEQ ID NO.8.

8. A chimeric antigen receptor targeting CD22 and CD19, wherein the chimeric antigen receptor is encoded by the nucleic acid molecule according to claim 1.

9. The chimeric antigen receptor according to claim 8, wherein an amino acid sequence of the chimeric antigen receptor is shown in SEQ ID NO.1, SEQ ID NO.2, SEQ ID NO.3, SEQ ID NO. NO.4, SEQ ID NO.15, SEQ ID NO.16, SEQ ID NO.17, and SEQ ID NO.18.

10. A vector, comprising the nucleic acid molecule according to claim 1.

11. A cell, comprising the nucleic acid molecule according to claim 1.

12. Application of the nucleic acid molecule according to claim 1 in preparation of anti-B-cell hematological tumor drugs, wherein B-cell hematological tumors are preferably B-cell lymphoma or acute B-lymphocytic leukemia.

13. Application of the chimeric antigen receptor according to claim 9 in preparation of anti-B-cell hematological tumor drugs, wherein B-cell hematological tumors are preferably B-cell lymphoma or acute B-lymphocytic leukemia.

14. Application of the vector according to claim 10 in preparation of anti-B-cell hematological tumor drugs, wherein B-cell hematological tumors are preferably B-cell lymphoma or acute B-lymphocytic leukemia.

15. Application of the cell according to claim 11 in preparation of anti-B-cell hematological tumor drugs, wherein B-cell hematological tumors are preferably B-cell lymphoma or acute B-lymphocytic leukemia.

16. The application according to claim 12, wherein the application is application in the preparation of the anti-B cell hematological tumor drugs for simultaneous expression of CD19 and CD22 or for recurrence or ineffectiveness of a patient after CD19 CAR-T treatment due to antigenic variation.

17. A pharmaceutical composition, comprising the nucleic acid molecule according to claim 1, and a pharmaceutically acceptable vector.

18. A pharmaceutical composition, comprising the chimeric antigen receptor according to claim 9, and a pharmaceutically acceptable vector.

19. Application of the pharmaceutical composition according to claim 17 in treatment of B-cell hematological tumors.

20. A method for treatment of B-cell hematological tumors, using the pharmaceutical composition according to claim 17.
